(19) United States
(12) Patent Application Publication
(10) Pub. No.: US 2021/0316124 A1
(43) Pub. Date: Oct. 14, 2021

(54) TETHERED ULTRASOUND DEVICES AND USES THEREOF

(71) Applicant: Suono Bio, Inc., Cambridge, MA (US)

(72) Inventors: Carl Schoellhammer, Cambridge, MA (US); Lisa Ricciardi, Cambridge, MA (US)

(21) Appl. No.: 17/282,066

(22) PCT Filed: Oct. 11, 2019
(86) PCT No.: PCT/US2019/055834

§ 371 Date: Apr. 1, 2021

Related U.S. Application Data
(60) Provisional application No. 62/744,232, filed on Oct. 11, 2018.
(60) Provisional application No. 62/744,224, filed on Oct. 11, 2018.
(60) Provisional application No. 62/755,065, filed on Nov. 2, 2018.
(60) Provisional application No. 62/757,842, filed on Nov. 9, 2018.

(51) Int. Cl. A61M 37/00; A61B 17/00

(52) U.S. Cl. CPC A61M 37/0092; A61M 2210/1042; A61B 2017/00296; A61B 17/00234

(57) ABSTRACT

The invention provides devices and methods for use with an endoscope to deliver an agent to an internal tissue of a subject. The devices and methods use transient acoustic cavitation to transfer an agent directly from a fluid to the tissue.

### CROSS-REFERENCE TO RELATED APPLICATIONS

This application claims the benefit of, and priority to, U.S. Provisional Application No. 62/744,224, filed Oct. 11, 2018; U.S. Provisional Application No. 62/744,232, filed Oct. 11, 2018; U.S. Provisional Application No. 62/755,065, filed September Nov. 2, 2018; and U.S. Provisional Application No. 62/757,842, filed Nov. 9, 2018, the contents of each of which are incorporated herein by reference.

### FIELD OF THE INVENTION

The invention relates generally to devices and methods for ultrasonic delivery of an agent to an internal tissue.

### BACKGROUND

Millions of people suffer from diseases for which no effective therapy exists. In many such cases, the molecular basis of the disease is known, but current reagents and delivery mechanisms are ineffective at modifying the activity of one or more molecular targets that cause the condition. So-called “undruggable” targets are associated with a wide array of serious diseases, including cancers, inflammatory diseases, and gastrointestinal disorders. Delivery of a drug through the gastrointestinal (GI) tract is desirable because it can be performed rapidly and with minimal invasion of the patient's body. However, conventional oral delivery, in which a patient swallows a solid or liquid formulation containing an active pharmaceutical ingredient (API), does not work for many drugs due to the acidic conditions and harsh digestive enzymes of the GI tract. Some therapeutic agents, such as biological therapeutics (“biologics”), which generally consist of large macromolecules, are poorly absorbed. Absorption may also be limited if the patient has a diarrhea, which minimizes the duration of transit of the drug through the GI tract.

Many biologics are administered intravenously, but this mode of administration has its own set of obstacles. For example, circulating biologics trigger the body's immune response, which results in destruction of the drug or its elimination from the body and nullifies its therapeutic benefit. Therefore, biologics are typically formulated in encapsulated structures or macromolecular complexes that allow them to evade detection by the immune system.

Consequently, in many instances, advances in the understanding of the biological mechanism of a disease have not been translated into effective therapies, and people continue to suffer from conditions that cannot be adequately treated.

### SUMMARY

The invention provides devices and methods for ultrasonic delivery of agents into gastrointestinal tissue of a patient. The devices include small ultrasound transducers that can be used in conjunction with endoscopes to deliver therapeutic agents rapidly into the upper GI tract. The devices promote transient acoustic cavitation of fluid in contact with gastrointestinal tissue to promote transfer of agents from the fluid into the tissue. Transfer occurs within a minute or less, so exposure of the agent to sonic energy and the harsh environment of the gut is minimized. The devices permit transfer of a broad range of agents, including organic small molecules and biological macromolecules. Use of the devices obviates the need to provide a drug, e.g., a biologic drug, in a protective formulation, such as an encapsulated structure or macromolecular complex. Consequently, the devices allow direct administration of pharmacological agents, such as biologics (e.g., nucleic acids such as siRNAs, mRNAs), and organic small molecules, in unmodified (i.e., native) forms that preserve their activity.

By providing improved delivery of therapeutic agents to internal tissue, such as the upper GI tract, the devices and methods unlock the therapeutic potential of a variety of agents. For certain agents that must be provided at high doses to achieve a therapeutic benefit with prior methods, methods of the invention achieve comparable therapeutic effect using greatly reduced dosages and/or less frequent administration. In other cases, the devices and methods allow therapeutically effective delivery of agents that previously had no clinically useful formulation or delivery mechanism. As a result, the invention allows for pharmaceutical intervention for many molecular targets that were previously considered “undruggable” and provides effective treatments for a multitude of diseases and disorders.

Methods and devices of the invention are also useful for vaccination. Nearly 10 million people die each year from infectious diseases. Many such deaths could have been prevented by vaccination, but immunization rates are sub-optimal due to a variety of factors. Many vaccines require administration of multiple doses over a period of months or years. Consequently, many patients fail to complete their course of vaccination, particularly in developing countries where many individuals do not have ready access to clinics. In addition, most vaccines are not absorbed reliably through the gastrointestinal tract and must be administered by injection with a hypodermic needle. Intramuscular or subcutaneous injection is painful and can frighten children, who often make up the patient population that vaccines are intended to benefit. Intradermal injection, which is used for the tuberculosis vaccine, is technically challenging to perform. Administration by injection also requires careful disposal of needles, and some developing countries lack the infrastructure to safely discard hazardous materials.

The invention provides methods of using ultrasound to deliver agents, such as antigens and other components of vaccines, directly to mucosal tissue. The methods involve applying ultrasound to antigen-containing fluid in contact with mucosal tissue, which is rich in immune cells. The ultrasound also causes transient cavitation of the fluid, and implosion of bubbles in the fluid propels the antigen into the mucosal tissue. In addition, application of ultrasound triggers activity of the immune cells. Consequently, the methods allow efficient delivery of the antigen to immune cells and stimulation of those cells to mount an effective response to the antigen. The methods are also useful for delivery of immunotherapeutic agents, such as antibodies, cytokines, and chemokines.

The ultrasound-based methods of immunization provided herein have several advantages over prior methods. First, delivery of an antigen to a mucosal tissue, such as the lining of the mouth, obviates the need for injection with a needle. Thus, ultrasound-based methods are more convenient for patients and reduce the amount of hazardous waste that must be discarded. The efficiency of antigen delivery and co-stimulatory effect of the ultrasound also result in improved immune response. Therefore, lower doses of the vaccine may be used, immunization may be achieved with a single administration, and more robust responses can be elicited. In addition, because antigens are delivered directly to immune cells, such as effector T cells, antigens need not be supplied in stabilizing or protective formulations, such as encapsulated formats or macromolecular complexes. Finally, ultrasound-based delivery allows immunization using toxoid vaccines in lieu of live-attenuated vaccines. Consequently, the methods of the invention facilitate development of vaccines that are safer and easier to store.

Methods and devices of the invention are also useful for treating wounds. Wounds, such as ulcers, burns, and lacerations, are serious medical problems. For example, burn wounds result in hospitalization of about 40,000 people, and diabetic foot ulcers are responsible for about 60,000 limb amputations in the United States each year. Open wounds are difficult to treat. Although evidence suggests that growth factors may promote wound healing, supplying growth factors to the wound site at therapeutic levels is problematic in clinical applications. Systemic delivery of growth factors carries the risk serious side effects and thus is only recommended for treatment of burn victims having burns covering greater than 40% of the body. Moreover, systemic delivery is ineffective in treating diabetic foot ulcers, which are usually accompanied by peripheral artery disease that limits delivery of blood-borne agents to the wound site. Topical application of compositions containing growth factors requires prolonged exposure of the wound to the therapeutic formulation to allow the growth factor to permeate the damaged tissue. Another difficulty is that certain clotting factors that promote healing of ulcers chemically degrade in the presence of light or other chemicals present in the delivery system. Consequently, such agents must be provided with a coating that protects them from degradation during delivery but is released from the active compound at the site of action.

The invention provides methods of using ultrasound energy to delivery therapeutic agents, such as growth factors, directly to wounds. Ultrasound waves are used to produce transient cavitation of a fluid containing the agent, and implosion of bubbles in the fluid propels the agent into the wound. Because transfer of the agent occurs directly from the fluid to the wounded tissue, it is rapid and efficient. Moreover, due to the short of time of delivery, the methods obviate the need for complicated formulations in which the agent is encapsulated or contained within a protective molecular complex.

The methods of the inventions overcome a variety of obstacles associated with prior methods of treating wounds, such as diabetic ulcers and burns. By delivering growth factors directly to the healing tissue, the methods avoid serious side effects caused by systemic delivery. Thus, the methods can be used to treat virtually any patient or wound type and need not be reserved for only the direst cases. In addition, the methods achieve superior wound penetration compared to methods that rely on passive diffusion because the ultrasound waves drive the growth factors into the wounded tissue. Furthermore, the simpler formulations that can be used with methods of the invention are easier and less expensive to produce and more stable during storage prior to use.

In an aspect, the invention provides devices for delivery of an agent to an internal tissue of a subject. The devices include an ultrasound transducer and an electrical conductor that operably couples the ultrasound transducer to a power source. Both the ultrasound transducer and the electrical conductor are configured to fit partly or completely within the lumen of the endoscope.

The ultrasound transducer has a maximum diameter small enough to allow the transducer to be passed through an endoscope. For example, the ultrasound transducer may have a maximum diameter of less than about 40 mm, less than about 20 mm, less than about 10 mm, less than about 8 mm, less than about 6 mm, less than about 5 mm, less than about 4 mm, less than about 3 mm, less than about 2 mm, or less than about 1 mm.

The device may include a cover that covers a portion of the ultrasound transducer. The cover may protect the ultrasound transducer from the milieu of the gut. For example, the cover may contain a material that is resistant to one or more of acid degradation and enzymatic degradation.

The device may include, or may be configured to connect to, a power source. The power source may be located outside the body of the subject. The electrical conductor may connect or be configured to connect the ultrasound transducer to the power source.

The electrical conductor may have a length sufficient to connect the transducer, while it is in a region of the gastrointestinal tract of a subject, to a power source located outside the subject. For example, the electrical conductor may have a length of at least about 100 mm, at least about 200 mm, at least about 300 mm, at least about 400 mm, at least about 500 mm, at least about 600 mm, at least about 800 mm, at least about 1 m, at least about 1.5 m, at least about 2 m, at least about 2.5 m, at least about 3 m, or at least about 4 m.

The device may contain a tube for delivering fluid from a fluid source to the ultrasound transducer. The fluid source may be located outside the body of the subject. The tube may run from the fluid source to the ultrasound transducer. The tube may be located completely or partly within, or configured to fit completely or partly within, the lumen of an endoscope. The proximal end of the tube may be connected to, or configured to be connected to, the fluid source. The distal end of the tube may contact the ultrasound transducer. The distal end of the tube may be positioned proximate to the ultrasound transducer.

In another aspect, the invention provides devices for delivery of an agent to an internal tissue of a subject. The devices include an ultrasound transducer configured to fit into the esophagus of a human and an electrical conductor that operably couples the ultrasound transducer to a power source.

The electrical conductor may be configured to fit partly or completely into the lumen of an endoscope.

The device may include a cover that covers a portion of the ultrasound transducer. The cover may protect the ultrasound transducer from the milieu of the gut. For example, the cover may contain a material that is resistant to one or more of acid degradation and enzymatic degradation.

The device may include, or may be configured to connect to, a power source. The power source may be located outside the body of the subject. The electrical conductor may connect or be configured to connect the ultrasound transducer to the power source.

The electrical conductor may have a length sufficient to connect the transducer, while it is in a region of the gastrointestinal tract of a subject, to a power source located outside the subject. For example, the electrical conductor may have a length of at least about 100 mm, at least about 200 mm, at least about 300 mm, at least about 400 mm, at least about 500 mm, at least about 600 mm, at least about 800 mm, at least about 1 m, at least about 1.5 m, at least about 2 m, at least about 2.5 m, at least about 3 m, or at least about 4 m.

The device may contain a tube for delivering fluid from a fluid source to the ultrasound transducer. The fluid source may be located outside the body of the subject. The tube may run from the fluid source to the ultrasound transducer. The tube may be located completely or partly within, or configured to fit completely or partly within, the lumen of an endoscope. The proximal end of the tube may be connected to, or configured to be connected to, the fluid source. The distal end of the tube may contact the ultrasound transducer. The distal end of the tube may be positioned proximate to the ultrasound transducer.

In another aspect, the invention provides methods of delivering an agent to an internal tissue of a subject. The methods include introducing via an esophagus of a subject an ultrasound transducer and a fluid containing an agent so that the fluid is proximate an internal tissue and the ultrasound transducer and delivering ultrasound energy from the ultrasound transducer into the fluid at a frequency to produce transient cavitation of the fluid, thereby propelling the agent into the internal tissue of the subject.

The methods may be used with device of the invention, such as those described above.

The methods may include inserting an endoscope into the subject. The fluid may be introduced via the endoscope. The fluid may be introduced by injection into the lumen of the endoscope. The fluid may be introduced via a tube housed completely or partly within the endoscope. The fluid may be introduced via a tube external to the endoscope.

In another aspect, the invention provides methods for immunizing a subject by introducing a fluid containing an antigen proximate a mucosal tissue of the subject and delivering ultrasound energy to the fluid at a frequency that causes the antigen to enter the mucosal tissue, thereby initiating an immune response that results in immunization of the subject.

In another aspect, the invention provides methods for immunizing a subject by introducing a fluid containing an antigen proximate a mucosal tissue of the subject and delivering ultrasound energy to the fluid at a frequency that results in an immune response and causes the antigen to enter the mucosal tissue, which also results in the immune response, in which the combination results in immunization of the subject.

In certain embodiments, the introducing and delivering steps are not repeated. In certain embodiments, the introducing step, the delivering step, or both are repeated.

The mucosal tissue may be gastrointestinal tissue. The gastrointestinal tissue may be buccal tissue, gingival tissue, labial tissue, esophageal tissue, gastric tissue, intestinal tissue, colorectal tissue, or anal tissue. The mucosal tissue may be nasal tissue or vaginal tissue.

The fluid may contain a formulation that prolongs release of the antigen into the mucosal tissue. The extended-release formulation may contain microparticles, nanoparticles, gels, liposomes, lipid vesicles, dendrimers, or virus-like particles. The extended release formulation may contain chitosan, gamma polyglutamic acid (γ-PGA), gelatin, hematin anhydride, hyaluronan, hyaluronic acid, latex, poly-(1,4-phenyleneacetone dimethylene thioketal), poly(alkylcyanoacrylate) (PACA), poly(lactic-co-glycolic acid) (PLGA), poly(methyl methacrylate) (PMMA), poly(phosphazenes), poly-alkyl-cyano-acrylates (PAC), polyanhydrides, polylactic acid (PLA), or poly-ε-caprolactone (PCL).

The antigen may be any agent that triggers an immune response. For example, the antigen may be a nucleic acid, a peptide, a polypeptide, a protein, an antibody, an organic molecule, a toxoid, or any combination thereof.

The antigen may have a minimum size. For example, the antigen may have a molecular weight of >100 Da, >200 Da, >500 Da, >1000 Da, >2000 Da, >5000 Da, >10,000 Da, >20,000 Da, >50,000 Da, or >100,000 Da.

The immune response may include activation of effector T cells. The effector T cells may be CD25⁺. The immune response may include recruitment of immune cells, dendritic cells, or both to a site at which the antigen entered the mucosal tissue.

The ultrasound energy may produce transient cavitation of the fluid. Implosion of bubbles in the fluid may propel the antigen into the mucosal tissue.

The ultrasound energy may be delivered at a frequency of from about 10 kHz to about 10 MHz. Preferably, the ultrasound energy is delivered at a frequency of less than 100 kHz. For example, the ultrasound energy may be delivered at a frequency of from about 20 kHz to about 60 kHz. The ultrasound energy may be delivered at a frequency of about 40 kHz.

The ultrasound energy may be delivered in a pulse. The pulse may be less than 20 minutes, less than 10 minutes, less than 5 minutes, or less than 2 minutes. The pulse may be from about 0.1 seconds to about 3 minutes. The pulse may be about 10 minutes, about 5 minutes, about 3 minutes, about 2 minutes, about 1 minute, about 30 seconds, about 20 seconds, or about 10 seconds. The pulse may include a duty cycle in which the ultrasound energy is applied intermittently or with gaps within the pulse. For example, the pulse may include two or more “on” periods separated by “off” periods. The “on” and “off” periods may be of any duration. For example and without limitation, the “on” and/or “off” periods may be about 10 milliseconds, about 20 milliseconds, about 50 milliseconds, about 0.1 seconds, about 0.2 seconds, about 0.5 seconds, about 1 second, about 2 seconds, about 5 seconds, about 10 seconds, about 20 seconds, about 30 seconds, about 1 minute, about 2 minutes or about 5 minutes.

The ultrasound energy may be selected so that it does not result in breakdown of a fraction or percentage of the antigen. For example, the ultrasound energy may result in breakdown of less than about 95% of the antigen, less than about 90% of the antigen, less than about 80% of the antigen, less than about 70% of the antigen, less than about 60% of the antigen, less than about 50% of the antigen, less than about 40% of the antigen, less than about 25% of the antigen, or less than about 10% of the antigen.

The ultrasound energy may be delivered from an ultrasound device that contains a horn. The horn may be in contact with the fluid. The device may contain a chamber that holds the fluid containing the antigen.

In another aspect, the invention provides methods of treating a target tissue of a subject by delivering ultrasound energy to a subject at a frequency that initiates an immune response directed at a target tissue of the subject and delivering ultrasound energy to a fluid at a frequency that causes an immunotherapeutic agent in the fluid to enter the target tissue of the subject, in which the combination of the immune response and the immunotherapeutic agent provides a treatment to the target tissue of the subject.

The target tissue may be a mucosal tissue, such as any of the tissues described above.

The immunotherapeutic agent may be or include an antibody, an antimicrobial, a chemokine, a cytokine, an imide drug, or an interleukin.

The ultrasound energy may produce transient cavitation of the fluid. Implosion of bubbles in the fluid may propel the immunotherapeutic agent into the target tissue. Implosion of bubbles in the fluid may propel the immunotherapeutic agent into immune cells, which may be in the target tissue.

The ultrasound energy may be delivered at a particular frequency or range of frequencies, such as any of those described above.

The ultrasound energy may be provided as one or more pulses, as described above.

The ultrasound energy may be selected so that it does not result in breakdown of a fraction or percentage of the immunotherapeutic agent. For example, the ultrasound energy may result in breakdown of less than about 95% of the immunotherapeutic agent, less than about 90% of the immunotherapeutic agent, less than about 80% of the immunotherapeutic agent, less than about 70% of the immunotherapeutic agent, less than about 60% of the immunotherapeutic agent, less than about 50% of the immunotherapeutic agent, less than about 40% of the immunotherapeutic agent, less than about 25% of the immunotherapeutic agent, or less than about 10% of the immunotherapeutic agent.

The ultrasound energy may be delivered from an ultrasound device that contains a horn. The horn may be in contact with the fluid. The device may contain a chamber that holds the fluid containing the immunotherapeutic agent.

In another aspect, the invention provides methods of treating a wound in a subject. The methods include providing a fluid containing an agent that promotes wound healing and delivering ultrasound energy to the fluid at a frequency to produce transient cavitation of the fluid to propel the agent into the wound of the subject, thereby treating the wound in the subject.

The agent may be any agent that promotes healing of a wound. The agent may be an analgesic, an antibiotic, an anticoagulant, an antimicrobial, an antioxidant, an antiseptic, a calcium channel blocker, a corticosteroid, a growth factor, honey, a methylxanthine, a nitric oxide donor, phenytoin, a prostacyclin analog, a retinoid, or a nucleic acid encoding any of the aforementioned agents. The anticoagulant may be heparin. The antimicrobial may be silver, iodine, chlorhexidine, or hydrogen peroxide. The antioxidant may be zinc. The calcium channel blocker may be diltiazem or nifedipine. The corticosteroid may be prednisolone. The growth factor may be CTGF/CCN2, an EGF family member, a FGF family member, G-CSF, GM-CSF, HGF, HGH, HIF, IGF, IL-1, IL-4, IL-8, KGF, lactoferrin, a PDGF, a TGF-β, or VEGF. The EFG family member may be amphiregulin (AR), betacellulin (BTC), epigen, epiregulin (EPR), heparin-binding EGF-like growth factor (HB-EGF), neuregulin-1 (NRG1), neuregulin-2 (NRG2), neuregulin-3 (NRG3), neuregulin-4 (NRG4), or transforming growth factor-α (TGF-α). The FGF family member may be FGF1, FGF2 (also called basic FGF or bFGF), FGF3, FGF4, FGF5, FGF6, FGF7, FGF8, FGF9, FGF10, FGF11, FGF12, FGF13, FGF14, FGF16, FGF17, FGF18, FGF19, FGF20, FGF21, FGF22, or FGF23. The PDGF may be PDGF AA, PDGF AB, or PDGF BB. The TGF-β may be TGF-β1, TGF-β2, or TGF-β3. The methylxanthine may be caffeine, aminophylline, 3-isobutyl-1-methylxanthine, paraxanthine, pentoxifylline, theobromine, or theophylline. The nitric oxide donor may be glyceryl trinitrate. The prostacyclin analog may be iloprost or cisaprost. The retinoid may be acitretin, adapalene, alitretinoin, bexarotene, etretinate, isotretinoin, retinal, retinol, tazarotene, or tretinoin (retinoic acid). The agent may be a component of a gene editing system, such as the CRISPR system.

The agent may have a minimum size. For example, the agent may have a molecular weight of >100 Da, >200 Da, >500 Da, >1000 Da, >2000 Da, >5000 Da, >10,000 Da, >20,000 Da, >50,000 Da, or >100,000 Da.

The wound may be any type of wound. For example, the wound may be an abrasion, a bedsore, a burn, a cosmetic blemish, a decubitus ulcer, a laceration, pressure gangrene, a surgical incision, or an ulcer. The wound may be an ulcer associated with diabetes.

The ultrasound energy may be delivered at a frequency of from about 10 kHz to about 10 MHz. Preferably, the ultrasound energy is delivered at a frequency of less than 100 kHz. For example, the ultrasound energy may be delivered at a frequency of from about 20 kHz to about 60 kHz. The ultrasound energy may be delivered at a frequency of about 40 kHz.

The ultrasound energy may be delivered in a pulse. The pulse may be less than 20 minutes, less than 10 minutes, less than 5 minutes, or less than 2 minutes. The pulse may be from about 0.1 seconds to about 3 minutes. The pulse may be about 10 minutes, about 5 minutes, about 3 minutes, about 2 minutes, about 1 minute, about 30 seconds, about 20 seconds, or about 10 seconds. The pulse may include a duty cycle in which the ultrasound energy is applied intermittently or with gaps within the pulse. For example, the pulse may include two or more “on” periods separated by “off” periods. The “on” and “off” periods may be of any duration. For example and without limitation, the “on” and/or “off” periods may be about 10 milliseconds, about 20 milliseconds, about 50 milliseconds, about 0.1 seconds, about 0.2 seconds, about 0.5 seconds, about 1 second, about 2 seconds, about 5 seconds, about 10 seconds, about 20 seconds, about 30 seconds, about 1 minute, about 2 minutes or about 5 minutes.

The ultrasound energy may be selected so that it does not result in breakdown of a fraction or percentage of the agent. For example, the ultrasound energy may result in breakdown of less than about 95% of the agent, less than about 90% of the agent, less than about 80% of the agent, less than about 70% of the agent, less than about 60% of the agent, less than about 50% of the agent, less than about 40% of the agent, less than about 25% of the agent, or less than about 10% of the agent.

The ultrasound energy may be delivered from an ultrasound device that contains a horn. The horn may be in contact with the fluid. The device may contain a chamber that holds the fluid containing the agent.

In an aspect, the invention provides methods of treating a diabetic ulcer in a subject. The methods include providing a fluid comprising an agent that promotes healing of a diabetic ulcer and delivering ultrasound energy to the fluid at a frequency to produce transient cavitation of the fluid to propel the agent into the diabetic ulcer of the subject, thereby treating the diabetic ulcer in the subject.

The agent may be any agent that promotes healing of an ulcer, such as any agent described above in relation to wound healing.

The subject may have a condition associated with the diabetic ulcer. For example, the condition may be cigarette smoking, diabetic neuropathy, edema, elderly status, a foot deformity, an infection, ischemia, limb amputation, peripheral vascular disease, poor glycemic control, or renal failure.

The ultrasound energy may be delivered at any frequency described above.

The ultrasound energy may be delivered in a pulse, as described above.

The ultrasound energy may be selected so that it does not result in breakdown of a fraction or percentage of the agent, as described above.

The ultrasound energy may be delivered from an ultrasound device having one or more of the features described above.

### BRIEF DESCRIPTION OF THE DRAWINGS

FIG. 1 is diagram of a device according to an embodiment of the invention.

FIG. 2 is diagram of a device according to an embodiment of the invention.

FIG. 3 is diagram of a device according to an embodiment of the invention.

FIG. 4 is diagram of a device according to an embodiment of the invention.

FIG. 5 shows an ultrasound device that can be used with methods of the invention.

FIG. 6 shows an ultrasound device that can be used with methods of the invention.

FIG. 7 shows an ultrasound device that can be used with methods of the invention.

FIG. 8A is perspective view of a device that can be used with methods of the invention.

FIG. 8B is a top view of the device shown in FIG. 8A.

FIG. 8C is a bottom view of the device shown in FIG. 8A.

FIG. 8D is a left side view of the device shown in FIG. 8A.

FIG. 8E is a right side view of the device shown in FIG. 8A.

FIG. 8F is a front view of the device shown in FIG. 8A.

FIG. 8G is a rear view of the device shown in FIG. 8A.

FIG. 8H is a section view of the device shown in FIG. 8A.

FIG. 9 is a shaded perspective view of a tip of a device that can be used with methods of the invention.

FIG. 10 is a lined perspective view of a tip of a device that can be used with methods of the invention.

FIG. 11 is a sectioned perspective section view of a tip of a device that can be used with methods of the invention.

FIG. 12 is a shaded bottom view of a tip of a device that can be used with methods of the invention.

FIG. 13 is a lined bottom view of a tip of a device that can be used with methods of the invention.

FIG. 14 is a side view of an ultrasound device that can be used with methods of the invention.

FIG. 15 is a view of a cartridge and device that can be used with methods of the invention.

FIG. 16 shows macroscopic (A) and microscopic (B) images of the use of ultrasound in the colon for the ultra-rapid delivery of material to a mucosal surface.

FIG. 17 is a graph showing delivery of fluorescently labeled latex particles of varying diameters into porcine colonic tissue ex vivo.

FIG. 18 is a Scanning Electron Microscopy (SEM) image of latex beads evenly disbursed over colonic tissue immediately after delivery with ultrasound.

FIG. 19 is a graph showing the fraction of the initial amount of material delivered into colonic tissue 24 hours after delivery for various fluorescently labeled permeants.

FIG. 20 is a schematic showing induction of dextran sulfate sodium-induced colitis and enema administration schedule.

FIG. 21 is a graph showing total fecal score for animals with acute colitis receiving various enemas.

FIG. 22 is graph showing histology scores of colonic tissue sections on Day 8.

FIG. 23 is a graph showing TNF protein levels from colonic tissue biopsies normalized by total protein content on Day 8.

FIG. 24 is a graph showing ova IgG titer levels 7-weeks after administration of ova alone or in combination with ultrasound.

FIG. 25 is a graph showing animal body weight after challenge with a lethal dose of Clostridium difficile spores on Day 1 normalized to the animals' weight pre-challenge.

### DETAILED DESCRIPTION

In modern biomedicine, identification of a pharmacological treatment for a disease typically occurs in the following sequence of steps: understanding of the molecular basis of the disease, identification of an agent that counteracts the aberrant molecular phenomenon, and development of a pharmaceutical composition or methodology that allows delivery of the agent to the appropriate tissue or location in the body to combat the disease. Each phase requires a substantial investment of time, human effort, and financial resources. However, delays during the third phase are particularly frustrating due to the sense that the solution is nearly in hand while people continue to suffer from the disease.

Myriad barriers can block or delay development of an agent that alters the activity of a disease-causing target in vitro into a useful therapeutic. For example, when organic small molecules are ingested, they may be modified by enzymes of the gut and liver during first-pass metabolism before they enter circulation. When this occurs, only a small percentage of the administered dose of the compound is available to affect its biological target. Many hydrophilic small molecules are unable to pass through cell membranes and thus must be formulated to facilitate cellular uptake. Conversely, hydrophobic small molecules often have poor solubility, a feature that must be overcome to allow such compounds to be distributed throughout the body via the circulatory system.

A distinct but overlapping set of problems faces molecules of biological origin. For example, inhibitory RNAs, such as siRNAs and miRNAs, hold great therapeutic promise due to the ease with which they can be adapted to affect different targets, but several technical obstacles must be overcome to make effective pharmacological agents from them. One problem is the susceptibility of these small RNA molecules to degradation by enzymes in serum and tissues. Another issue is that siRNAs exert their effects inside cells but do not enter cells readily. Consequently, siRNA-based therapeutics typically contain siRNA molecules that are encapsulated or complexed with other macromolecules to promote cellular uptake. A broader problem that confronts not only interfering RNAs but all biological therapeutics, such as proteins, antibodies, and nucleic acids for gene therapy, is their immunogenicity. When foreign macromolecules enter circulation, they are recognized by the immune system as foreign and destroyed and/or eliminated. Thus, to retain their efficacy, biologics must be modified in a way that masks their immunogenic elements without interfering with their biological function.

The invention overcomes the aforementioned issues by providing devices and methods for direct delivery of an active pharmacological agent to an internal tissue, such as the upper GI tract, of a subject. The devices include ultrasound transducers for use in conjunction with an endoscope. When fluid containing the agent is placed proximate to the internal tissue and the ultrasound transducer, a signal from the ultrasound horn causes transient acoustic cavitation of fluid that transfers the agent into the tissue. Because the transfer occurs directly from the fluid to tissue, the agent need not be provided in a special formulation, such as an encapsulated format or macromolecular complex with other molecules that are not pharmacologically active. In addition, the use of transient cavitation allows the transfer to occur rapidly, typically within minutes. One benefit of the rapid transfer is that exposure of the agent to ultrasonic vibration, which can cause unfolding or breakdown, is minimized and the activity of the agent is preserved. The rapid transfer also makes the procedure easier and less burdensome for patients and physicians.

Although vaccination represents one of the greatest medical advances of the 20th century, infectious diseases continue to kill millions of people worldwide each year well into the 21st century. Despite the existence of effective vaccines against many pathogens, immunization rates even in developed countries remain below the levels needed for widespread protection. Many vaccines must be administered by intramuscular injection to young children, who find the process frightening and painful. In addition, effective vaccination against many diseases requires multiple doses over the course of months or years, and many patients fail to complete the required series. Logistical problems further hamper immunization in developing countries. For example, the need for refrigerated storage of vaccine doses and safe disposal of used needles requires vaccines to be delivered in a clinical setting, but traveling to a hospital or clinic takes hours or days for many residents of rural countries.

The invention overcomes many of the barriers that prevent compliance with vaccination protocols by providing quick, effective methods of immunization. The methods use ultrasound waves to produce transient cavitation in a fluid containing an antigen, and implosion of bubbles in the fluid propels the antigen into mucosal tissue. By targeting mucosal tissue that is rich in immune cells, the methods convey antigens directly to the critical cell population. In addition, the ultrasound energy itself stimulates activity of immune cells, which intensifies their response to the delivered antigen. Consequently, immunization against some pathogens can be achieved in a single dose. In addition, by using simple ultrasound devices, the methods do not produce large volumes of hazardous waste materials. Thus, the methods solve many of the problems that impede widespread immunization using prior methods.

The invention also provides methods of ultrasonic delivery of immunotherapies, such as antibodies, chemokines, and cytokines. The methods also deploy ultrasound waves to deliver an agent to mucosal tissue by transient cavitation of fluid containing the agent.

The invention provides effective methods for treatment of wounds, such diabetic foot ulcers. Ulcers represent a major complication of diabetes mellitus and in many cases lead to amputation of the affected limb. Diabetes is often accompanied by narrowing of peripheral arteries, which contributes to the development of ulcers. In addition, the decreased blood supply prevents effective delivery of therapeutic agents in the bloodstream to the wound site, making ulcers difficult to treating using systemic therapies. On the other hand, topical delivery of therapeutic agents is limited by the need for the treatment method to avoid causing further structural damage to the wound. Such gentle methods result in slow delivery and require prolonged exposure of the therapeutic agent to the ulcer.

The invention overcomes these problems by using ultrasound energy to rapidly and efficiently deliver therapeutic agents, such as growth factors, from a fluid to a wound. Application of ultrasound waves causes transient formation of bubbles in the fluid, and collapse of the bubbles produces sufficient force to drive agents from the fluid into the wounded tissue. At the same time, the force is not so great as to cause damage to the tissue or disrupt the healing process.

### Ultrasound Devices

The methods of the invention may be performed with any ultrasound device that can induce transient cavitation. Transient cavitation can be achieved using a variety of ultrasound probe configurations, including axial and radial emission. Examples of suitable ultrasonic devices are described in, for example, U.S. Publication No. 2018/0055991 and co-pending, co-owned U.S. Application No. 62/701,408, the contents of each of which are incorporated herein by reference.

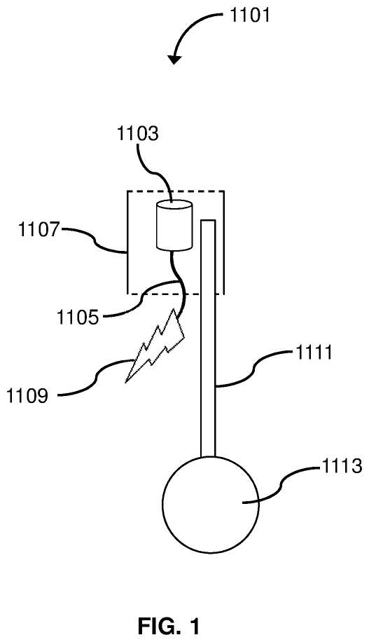

FIG. 1 is diagram of a device 1101 according to an embodiment of the invention. The device 1101 includes an ultrasound transducer 1103 coupled to an electrical conductor 1105. The device is configured to fit into an endoscope 1107. The electrical conductor 1105 is connected to a power source 1109. During use of the device 1101, the ultrasound transducer 1103, electrical conductor 1105, and endoscope 1107 are inserted into the body of the subject, while the power source 1109 may remain external to the subject's body. The electrical conductor 1105 may also serve as a tether that retains the ultrasound transducer 1103 and permits retraction of the device 1101 after use. The device 1101 may include a tube 1111 connected to a fluid source 1113 that allows delivery of the fluid to the ultrasound transducer 1103 during use. The distal end of the tube may be in contact with, or proximate to, the ultrasound transducer 1103. As shown here, the tube 1111 is configured to fit within the endoscope 1107. Alternatively, fluid may be injected directly into the lumen of the endo scope 1107.

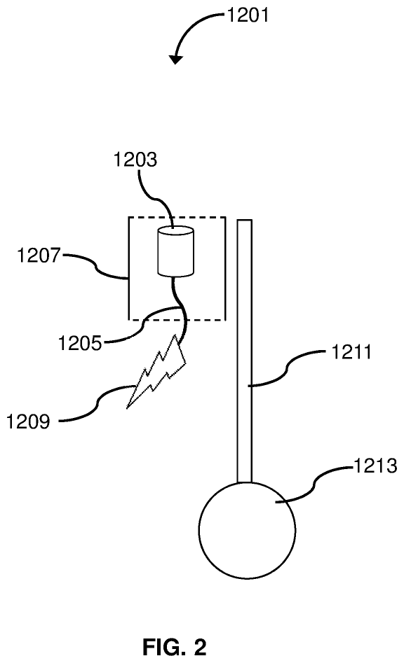

FIG. 2 is diagram of a device 1201 according to an embodiment of the invention. The device 1201 includes an ultrasound transducer 1203 coupled to an electrical conductor 1205. The device is configured to fit into an endoscope 1207. The electrical conductor 1205 is connected to a power source 1209. During use of the device 1201, the ultrasound transducer 1203, electrical conductor 1205, and endoscope 1207 are inserted into the body of the subject, while the power source 1209 may remain external to the subject's body. The electrical conductor 1205 may also serve as a tether that retains the ultrasound transducer 1103 and permits retraction of the device 1201 after use. The device 1201 may include a tube 1211 connected to a fluid source 1213 that allows delivery of the fluid to the ultrasound transducer 1103 during use. The distal end of the tube may be in contact with, or proximate to, the ultrasound transducer 1103. As shown here, the tube 1211 is external to the endoscope 1107. Alternatively, fluid may be injected directly into the lumen of the endoscope 1107.

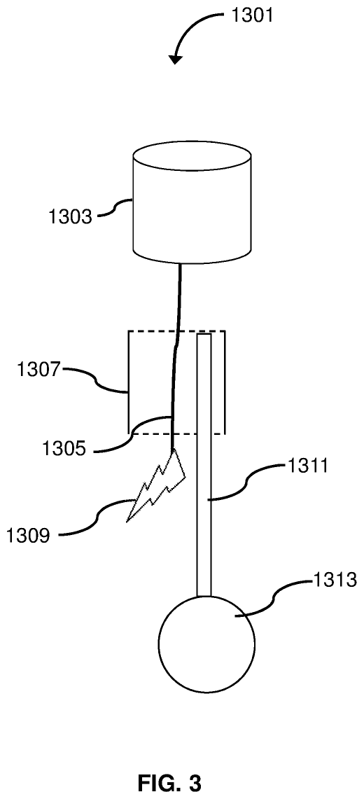

FIG. 3 is diagram of a device 1301 according to an embodiment of the invention. The device 1301 includes an ultrasound transducer 1303 coupled to an electrical conductor 1305. The ultrasound transducer 1303 is configured to be placed down the esophagus of a subject. The electrical conductor may be configured to fit partly or completely within an endoscope 1307. The electrical conductor 1305 is connected to a power source 1309. During use of the device 1301, the ultrasound transducer 1303, electrical conductor 1305, and endoscope 1307 are inserted into the esophagus of the subject, while the power source 1309 may remain external to the subject's body. The electrical conductor 1305 may also serve as a tether that retains the ultrasound transducer 1103 and permits retraction of the device 1301 after use. The device 1301 may include a tube 1311 connected to a fluid source 1313 that allows delivery of the fluid to the ultrasound transducer 1303 during use. The distal end of the tube may be in contact with, or proximate to, the ultrasound transducer 1103. As shown here, the tube 1311 is configured to fit within the endoscope 1307. Alternatively, fluid may be injected directly into the lumen of the endoscope 1307.

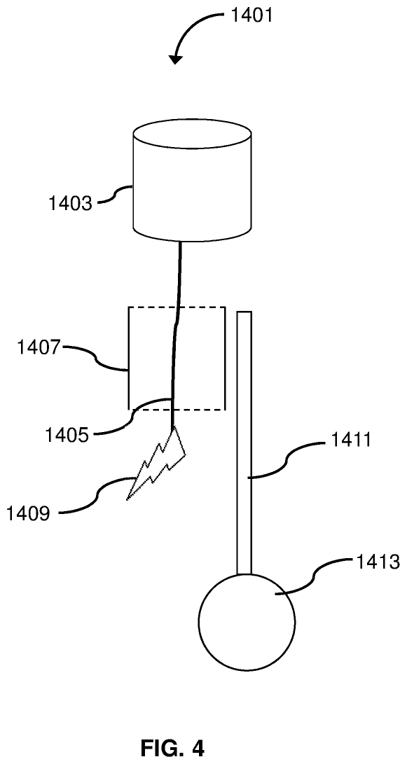

FIG. 4 is diagram of a device 1401 according to an embodiment of the invention. The device 1401 includes an ultrasound transducer 1403 coupled to an electrical conductor 1405. The ultrasound transducer 1403 is configured to be placed down the esophagus of a subject. The electrical conductor may be configured to fit partly or completely within an endoscope 1407. The electrical conductor 1405 is connected to a power source 1409. During use of the device 1401, the ultrasound transducer 1403, electrical conductor 1405, and endoscope 1407 are inserted into the esophagus of the subject, while the power source 1409 may remain external to the subject's body. The electrical conductor 1405 may also serve as a tether that retains the ultrasound transducer 1103 and permits retraction of the device 1401 after use. The device 1401 may include a tube 1411 connected to a fluid source 1413 that allows delivery of the fluid to the ultrasound transducer 1103 during use. The distal end of the tube may be in contact with, or proximate to, the ultrasound transducer 1403. As shown here, the tube 1411 is configured to fit within the endoscope 1407. Alternatively, fluid may be injected directly into the lumen of the endoscope 1407.

Suitable ultrasound transducers for any of the devices above include those sold under the trade names VCX 500 and VCX 130 (Sonics & Materials, Inc.; Newtown, Conn.). Suitable ultrasound transducers 103 and are described in, for example, Schoellhammer, C. M., Schroeder, A., Maa, R., Lauwers, G. Y., Swiston, A., Zervas, M., et al. (2015) Ultrasound-mediated gastrointestinal drug delivery, Science Translational Medicine, 7(310), 310ra168-310ra168, doi: 10.1126/scitranslmed.aaa5937; Schoellhammer, C. M & Traverso, G., Low-frequency ultrasound for drug delivery in the gastrointestinal tract. Expert Opinion on Drug Delivery, 2016, doi: 10.1517/17425247.2016.1171841; Schoellhammer C. M., et al., Ultrasound-mediated delivery of RNA to colonic mucosa of live mice. Gastroenterology, 2017, doi: 10.1053/j.gastro.2017.01.002; and U.S. Publication Nos. 2014/0228715 and 2018/0055991, the contents of each of which are incorporated herein by reference.

Any of the devices described above may contain a cover that encloses part or all of the ultrasound transducer to protect it from the harsh conditions of the digestive tract. Preferably, the cover is made from a material that is resistant to degradation from acidic conditions and from digestive enzymes, such as those found in the stomach and intestines. The cover should also be made of acoustically transparent material that allows transmission of ultrasound energy from the ultrasound transducer to the fluid.

Any suitable energy source may be used in conjunction with the devices described above. For example and without limitation, the energy source may an electricity source, battery, generator, or the like.

Any of the devices described above may contain one or more safety features that prevent events that could be harmful to the subject, such as excessive heating of the fluid or transmission of electrical signal. For example, the device may contain a thermocouple that couples the fluid distal end of the endoscope to the ultrasound transducer. The thermocouple can provide negative feedback to inactivate that ultrasound transducer when the temperature of the fluid and/or tissue becomes elevated beyond a threshold value. The device may contain a circuit breaker that is coupled to the ultrasound transducer and terminates a signal to the ultrasound transducer in response to a stimulus. For example and without limitation, the circuit breaker may terminate the signal to the ultrasound transducer after a certain period of time, when the temperature of the fluid and/or tissue becomes elevated beyond a threshold value, or when the electrical circuit containing the ultrasound transducer reaches a threshold value of resistivity or voltage.

The device may include, or be operably connected to, a control unit. The control unit may include one or more of an input mechanism, an output mechanism, a logic board, and an ultrasound driver board. For example and without limitation, the input mechanism may include buttons, switches, a keyboard, or the like. For example and without limitation, the output mechanism may provide a visual, audible, tactile, or vibrational signal.

The device may include an illumination source that illuminates the opening at the distal end of the endoscope. The illumination source may be a light, such as an incandescent light, light-emitting diode, or laser.

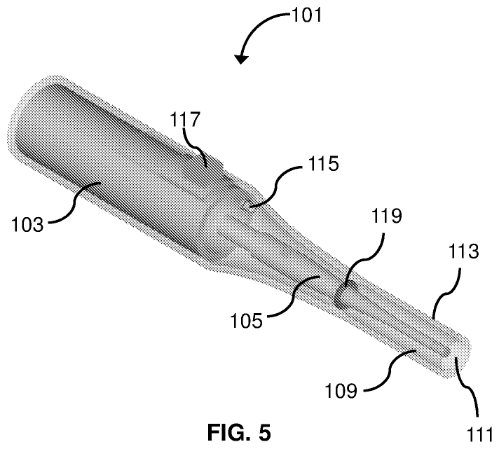

FIG. 5 shows an ultrasound device 101 that can be used with methods of the invention. The device 101 includes an ultrasound transducer 103 coupled to an ultrasound horn 105. The ultrasound horn 105 extends into a fluid chamber 109 that is fluidically separated from the ultrasound transducer 103. The fluid chamber 109 includes at least one opening 111 that can be positioned against a mucosal tissue. Suitable ultrasound transducers 103 include those sold under the trade names VCX 500 and VCX 130 (Sonics & Materials, Inc.; Newtown, Conn.). Suitable ultrasound transducers 103 and ultrasound horns 105 are described in, for example, Schoellhammer, C. M., Schroeder, A., Maa, R., Lauwers, G. Y., Swiston, A., Zervas, M., et al. (2015) Ultrasound-mediated gastrointestinal drug delivery, Science Translational Medicine, 7(310), 310ra168-310ra168, doi: 10.1126/scitranslmed.aaa5937; Schoellhammer, C. M & Traverso, G., Low-frequency ultrasound for drug delivery in the gastrointestinal tract. Expert Opinion on Drug Delivery, 2016, doi: 10.1517/17425247.2016.1171841; Schoellhammer C. M., et al., Ultrasound-mediated delivery of RNA to colonic mucosa of live mice. Gastroenterology, 2017, doi: 10.1053/j.gastro.2017.01.002; and U.S. Publication Nos. 2014/0228715 and 2018/0055991, the contents of each of which are incorporated herein by reference.

The device 101 may include an enclosure 113 that surrounds a portion of the fluid chamber 109. The enclosure 113 may also surround a portion of the ultrasound transducer 103, as shown. The enclosure 113 may dampen sound produced by the device 101. For example, the enclosure 113 may inhibit transmission of sound waves in directions other than toward the opening 111 of the fluid chamber 109.

The device 101 may include an illumination source 115 that illuminates the opening 111 of the fluid chamber 109. The illumination source 115 may be any type that facilitates positioning of the opening 111 of the device 101 against a mucosal tissue. The illumination source 115 may be a light, such as an incandescent light, light-emitting diode, or laser.

The device 101 may include an actuator 117 that activates the ultrasound transducer 103. The actuator 117 may be a binary on/off switch, or it may have a range of power settings for the ultrasound transducer 103.

The device 101 may include a separator 119 that separates the fluid chamber 109 from the section of the device 101 that houses the ultrasound transducer 103. The separator may be a gasket or O-ring, as shown. In embodiments in which the ultrasound horn 105 extends linearly into the fluid chamber, as shown, the separator 119 should be positioned at a node of vibration of the ultrasound horn 105.

The fluid chamber 109 may be contained in a tip of the device 101, such as a disposable tip. For example, the tip may comprise a cartridge that is fastened onto the front end of the device 101. The cartridge may contain a film or protective that is punctured by the ultrasound horn 105 when the cartridge is placed on the front of the device, thereby allowing the ultrasound horn 105 to contact the liquid in the fluid chamber 109. Such an arrangement allows the device 101 to be used repeatedly merely by replacing the cartridge at the tip and also facilitates preparation and storage of the liquid and antigen or immunotherapeutic agent within the fluid chamber.

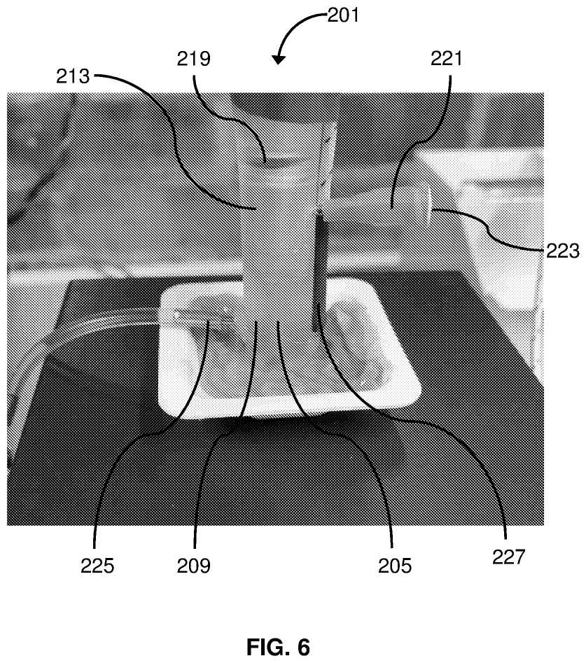

FIG. 6 shows an ultrasound device 201 according to an embodiment of the invention. The device 201 includes an ultrasound horn 205 that extends into a fluid chamber 209. As described above, the device 201 may include an enclosure 213 that surrounds a portion of the fluid chamber 209 and/or a separator 219 that separates the fluid chamber 209 from the ultrasound transducer.

The enclosure 213 may contain a vent 221 to allow the exchange of gas between the fluid chamber 209 and the ambient air. The vent 221 may include exhaust microchannels. Exhaust microchannels facilitate filling the fluid chamber with liquid by permitting release of gas from the chamber. The vent 221 with exhaust microchannels may be positioned on the enclosure at any point that allows upward release of gas when the device is oriented to deliver the antigen or immunotherapeutic agent to the subject, thus avoiding the need to hold the device with the tip downward during use. Additionally or alternatively, the vent 221 may contain a membrane 223 that is permeable to gas but impermeable to liquid. Many gas-permeable, liquid-impermeable materials are known in the art and described in, for example, U.S. Pat. Nos. 3,953,566; 4,152,482; 4,391,873; 4,500,328; 4,520,056; 4,772,508; 4,957,522; 5,522,769; and 6,676,871; and U.S. Publication No. 2010/0107878. For example and without limitation, the membrane may contain one or more of ethyl cellulose, ethyl/vinyl acetate, ethylene/acrylic acid copolymers, ethylene/alpha-olefin copolymers, ethylene/ethyl acrylate and, ethylene/methyl acrylate, fluoropolymers., fluorosilicone derived from, fluorovinylmethylsilicone, homopolymer polyethylenes, metallocene polypropylenes, nitrate butadiene rubber (NBR), nitrile rubber, poly(4-methyl-1-pentene), polydimethylsiloxane, polydimethylsiloxane, polyethylene, polyimides, polyisoprene, polyoctenamer, polyolefin, polyphenylvinylmethylsiloxane, polypropylene materials, polypropylene, polyethylene, polypropylenes, polytetrafluoroethylene, polyurethanes, polyvinylmethylsiloxane, propylene/alpha-olefin copolymers, propylene/ethylene copolymer, radical low-density polyethylenes, tetrafluoroethylene-(perfluoroalkyl) vinyl ether copolymer (PFA), and tetrafluoroethylene-hexafluoropropylene copolymer (FEP).

The enclosure 213 may contain a port 225 that fluidically connects the fluid chamber to a fluid source. The fluid source may be a component of the device 201 or may be functionally coupled to the device 201.

The device 201 may contain one or more safety features that prevent events that could be harmful to the subject, such as excessive heating of the fluid or transmission of electrical signal. For example, the device 201 may contain a thermocouple 227 that couples the fluid chamber to the ultrasound transducer. The thermocouple 227 can provide negative feedback to inactivate that ultrasound transducer when the temperature of the fluid and/or tissue becomes elevated beyond a threshold value. The device 201 may contain a circuit breaker that is coupled to the ultrasound transducer and terminates a signal to the ultrasound transducer in response to a stimulus. For example and without limitation, the circuit breaker may terminate the signal to the ultrasound transducer after a certain period of time, when the temperature of the fluid and/or tissue becomes elevated beyond a threshold value, or when the electrical circuit containing the ultrasound transducer reaches a threshold value of resistivity or voltage.

The device 201 may include, or be operably connected to, a control unit. The control unit may include one or more of an input mechanism, an output mechanism, a logic board, and an ultrasound driver board. For example and without limitation, the input mechanism may include buttons, switches, a keyboard, or the like. For example and without limitation, the output mechanism may provide a visual, audible, tactile, or vibrational signal.

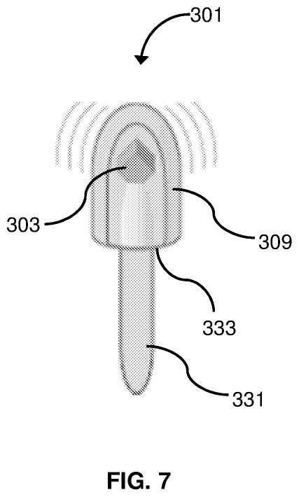

FIG. 7 shows another ultrasound device 301 that can be used with methods of the invention. The device 301 may have generally have a “lollipop” shape. Thus, the device 301 may have a handle 331 that can be held by the subject or a person administering the antigen or immunotherapeutic agent to the subject and a tip 333 that can be placed in an oral cavity of the subject. The ultrasonic transducer 303 is contained within a central portion of the tip 333, and the fluid chamber 309 comprises an exterior portion of the tip 333. An opening may be positioned at any point on an exterior surface of the fluid chamber 309 to facilitate contact of the fluid with the mucosal tissue.

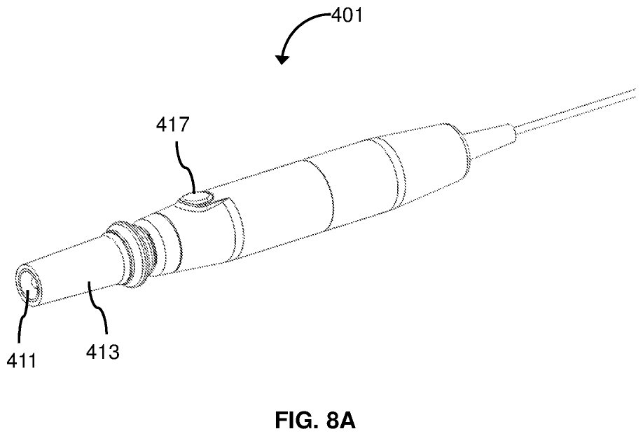

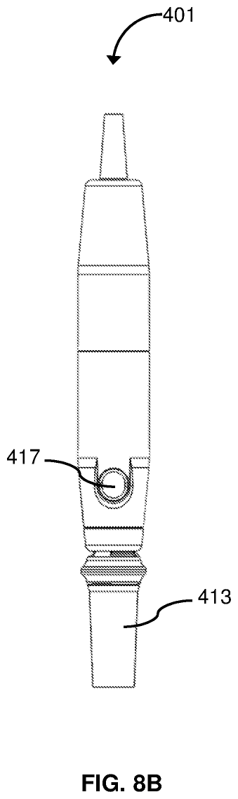

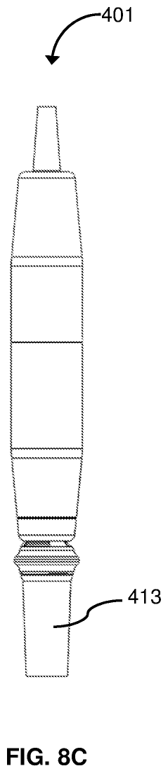

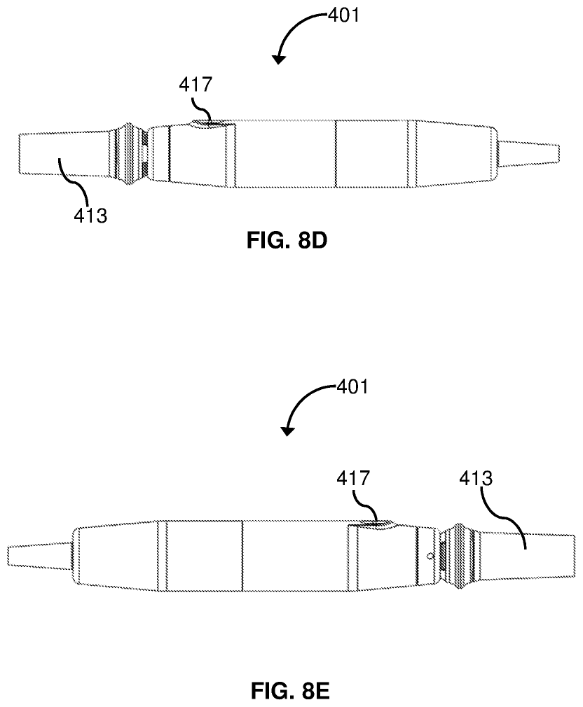

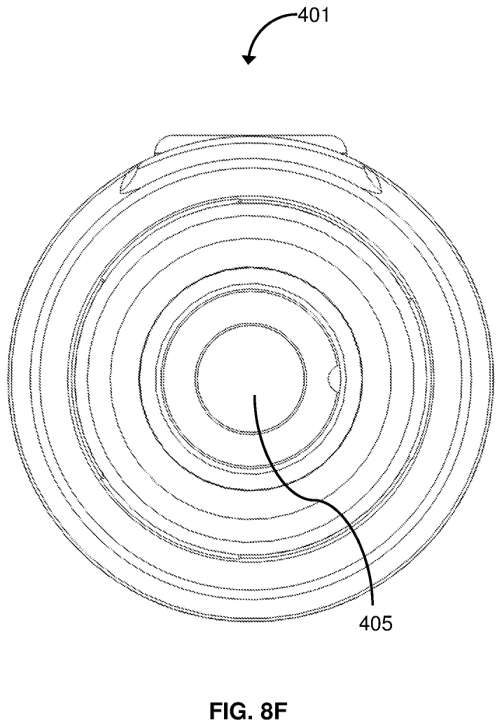

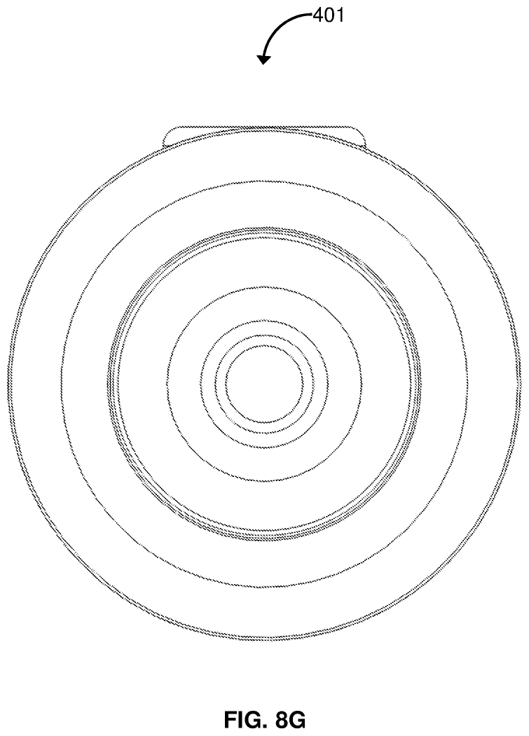

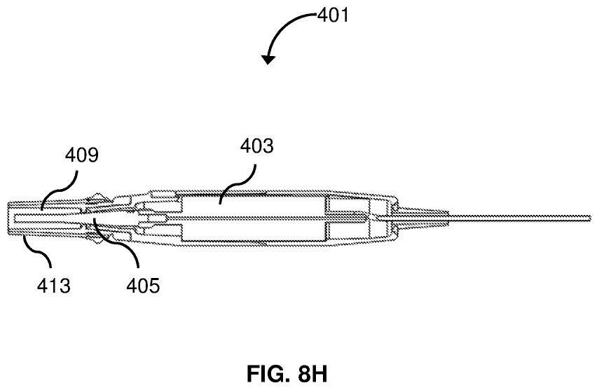

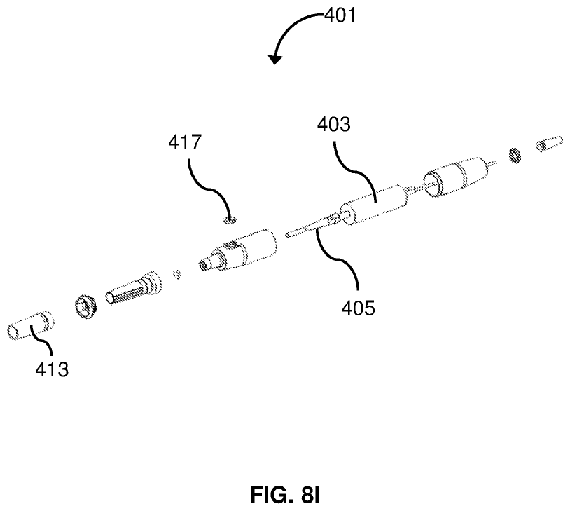

FIG. 8A is perspective view of a device that can be used with methods of the invention. The actuator 417, enclosure 413, and opening 411 are indicated in this view.

FIG. 8B is a top view of the device shown in FIG. 8A. The actuator 417 and enclosure 413 are indicated in this view.

FIG. 8C is a bottom view of the device shown in FIG. 8A. The enclosure 413 is indicated in this view.

FIG. 8D is a left side view of the device shown in FIG. 8A. The actuator 417 and enclosure 413 are indicated in this view.

FIG. 8E is a right side view of the device shown in FIG. 8A. The actuator 417 and enclosure 413 are indicated in this view.

FIG. 8F is a front view of the device shown in FIG. 8A. The ultrasound horn 405 is indicated in this view.

FIG. 8G is a rear view of the device shown in FIG. 8A.

FIG. 8H is a section view of the device shown in FIG. 8A. The enclosure 413, fluid chamber 409, ultrasound horn 405, and ultrasound transducer 403 are indicated in this view.

FIG. 8I is an exploded view of the device shown in FIG. 8A. The enclosure 413, actuator 413, ultrasound horn 405, and ultrasound transducer 403 are indicated in this view.

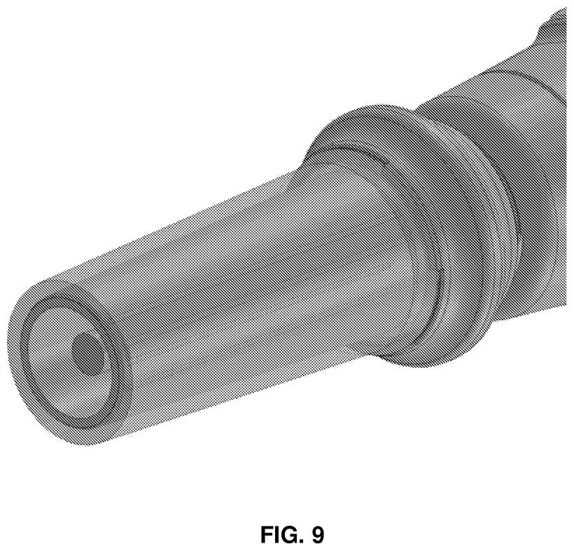

FIG. 9 is a shaded perspective view of a tip of a device that can be used with methods of the invention.

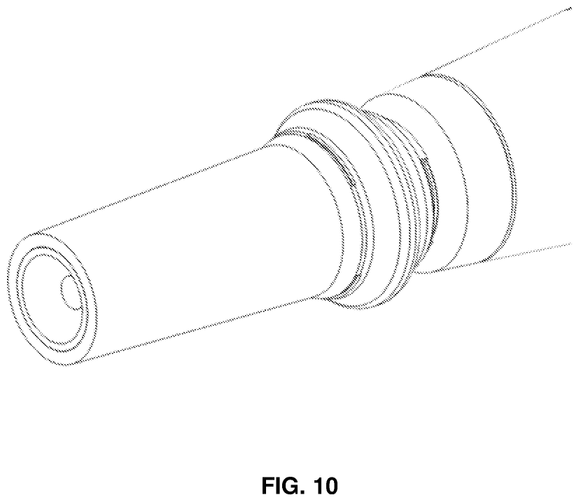

FIG. 10 is a lined perspective view of a tip of a device that can be used with methods of the invention.

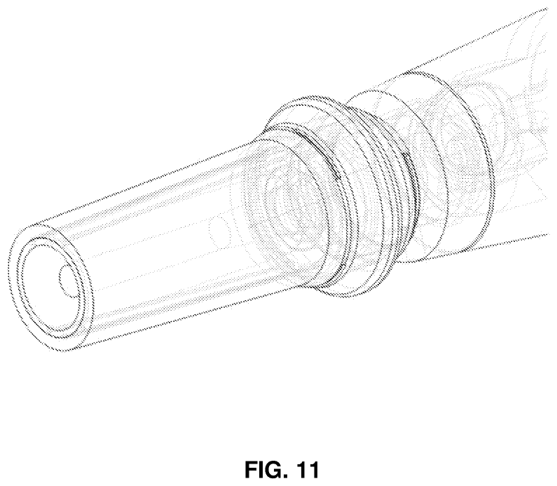

FIG. 11 is a sectioned perspective section view of a tip of a device that can be used with methods of the invention.

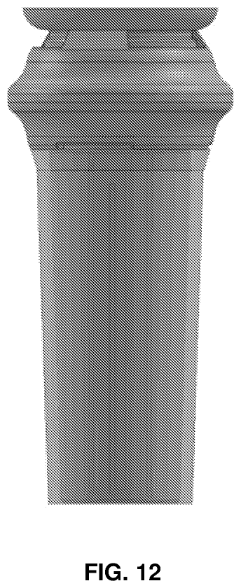

FIG. 12 is a shaded bottom view of a tip of a device that can be used with methods of the invention.

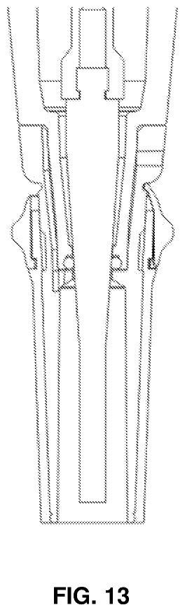

FIG. 13 is a lined bottom view of a tip of a device that can be used with methods of the invention.

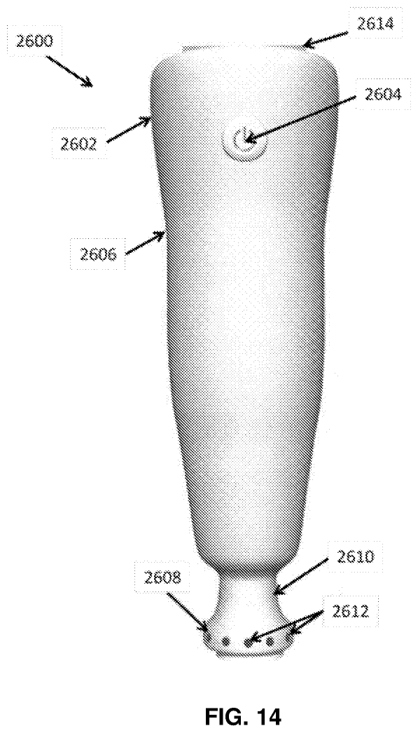

FIG. 14 is a side view of a reusable hand-held, ultrasound emitting ultrasound device 2600 that can be used with methods of the invention. The device 2600 includes a housing 2602, which may be cylindrical in shape with a taper down the length of the device. The housing 2602 may include or define a power control (e.g., a button or switch) 2604 to turn the device to be on or off. The housing may include a concave region 2606 that supports holding, positioning, and/or gripping the device by a user. The power control 2604 and concave region 2606 may be located toward the proximal end of the device, whereas the opposite distal end includes a tip 2608 for positioning near the mucosal tissue. The proximal base of the tip 2608 may include a concave region 2610 for creating a seal around the tip 2608 and the rectum. The tip 2608 may define at least one opening 2612 for delivering a substance from inside the device. The at least one opening 2612 may be oriented radially or axially to device 2600. Device 2600 also may define a port 2614 for receiving a cartridge containing a substance for delivery from the device.

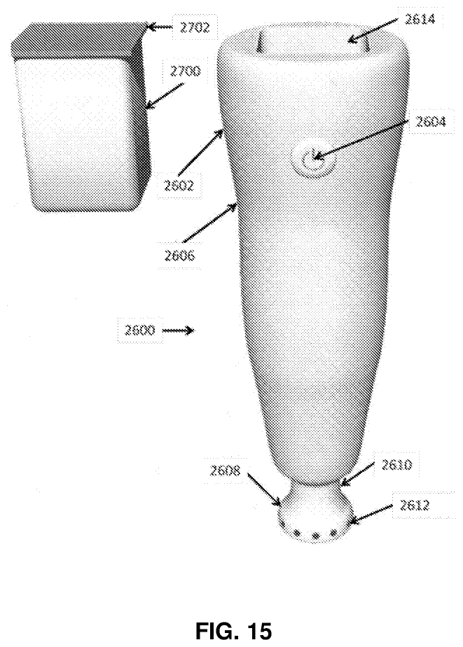

FIG. 15 shows a cartridge 2700 for use with device 2600 according to methods of the invention. The cartridge 2700 may be replaceable. The cartridge 2700 may have a top ridge 2702 to allow for the cartridge to remain in place once inserted into the device. Alternatively or in addition to a cartridge, device 2600 may receive the substance from an exterior container 2800, which may be compressible, thereby allowing the user to manually expel the substance by compressing the container 2800. The container 2800 may be connected to the device 2600 with, for example, flexible tubing 2802.

The housing of the device, excluding the tip, may include a rubberized coating or material that allows the user to hold the device securely. The tip may include a frictionless or low friction coating or material that allows for smooth insertion of the tip into the rectum. The housing, tip, or both may be water-resistant or waterproof for cleaning. The dimensions of the device include a length of about 14 cm to about 40 cm, a diameter of about 4 cm to about 6 cm at the top of the device, and a diameter of about 1 cm to about 3 cm at the tip of the device.

Other ultrasound devices capable of producing transient cavitation in a fluid are described in, for example, U.S. Pat. Nos. 7,377,905 and 8,202,369 and U.S. Publication No. 2004/0092921, the contents of each of which are incorporated herein by reference.

The ultrasound device may be reusable. Alternatively or additionally, the ultrasound device or a component of it may be disposable.

### Ultrasound Delivery Methods

The invention provides methods of delivering agents to tissue of a subject using devices of the invention. The methods include introducing placing an ultrasound transducer and fluid containing one or more agents so that the fluid is proximate to, or in contact with, both the tissue and the ultrasound transducer. The methods further include delivering ultrasound energy from the transducer to produce transient cavitation of the fluid, which propels the agent into the tissue.

The methods include delivering ultrasound energy to the fluid at a frequency that produces bubbles within the fluid and causes transient cavitation of the bubbles. Gentle implosion of the bubbles produces shock waves that permeabilize cells and propel the agent from the fluid into the tissue. The use of ultrasound to cause transient cavitation to deliver agents to tissue is described in, for example, Schoellhammer, C. M., Schroeder, A., Maa, R., Lauwers, G. Y., Swiston, A., Zervas, M., et al. (2015). Ultrasound-mediated gastrointestinal drug delivery. Science Translational Medicine, 7(310), 310ra168-310ra168, doi: 10.1126/scitranslmed.aaa5937; Schoellhammer, C. M & Traverso, G., Low-frequency ultrasound for drug delivery in the gastrointestinal tract. Expert Opinion on Drug Delivery, 2016, doi: 10.1517/17425247.2016.1171841; Schoellhammer C. M., et al., Ultrasound-mediated delivery of RNA to colonic mucosa of live mice, Gastroenterology, 2017, doi: 10.1053/j.gastro.2017.01.002; and U.S. Publication Nos. 2014/0228715 and 2018/0055991, the contents of each of which are incorporated herein by reference.

The frequency of the ultrasound energy may be between 10 kHz and 10 MHz. Preferably, the frequency of the ultrasound energy is less than less than 100 kHz. For example and without limitation, the frequency may be from about 20 kHz to about 100 kHz, from about 20 kHz to about 80 kHz, from about 20 kHz to about 60 kHz, or from about 30 kHz to about 50 kHz. The frequency may about 20 kHz, about 30 kHz, about 40 kHz, about 50 kHz, or about 60 kHz.

In some embodiments, the ultrasound energy may be delivered as a pulse, i.e., it may be delivered over a brief, finite period in order to minimize damage to the agent being delivered by the ultrasound energy. For example and without limitation, the pulse may be less than 20 minutes, less than 10 minutes, less than 5 minutes, or less than 10 minutes. For example and without limitation, the pulse may be from about 10 seconds to about 3 minutes. The pulse may be about 10 minutes, about 5 minutes, about 3 minutes, about 3 minutes, about 1 minute, about 30 seconds, about 20 seconds, or about 10 seconds.

The parameters of the ultrasound pulse, such as the frequency and/or duration, may be selected so that damage to the agent is limited to a certain fraction or percentage of the agent. For example and without limitation, the ultrasound energy may result in breakdown of less than about 95% of the agent, less than about 90% of the agent, less than about 80% of the agent, less than about 70% of the agent, less than about 60% of the agent, less than about 50% of the agent, less than about 40% of the agent, less than about 25% of the agent, or less than about 10% of the agent.

The parameters of the ultrasound pulse, such as the frequency and/or duration, may be selected so that at least a minimum amount of the agent is transferred to the tissue. For example and without limitation, the ultrasound energy may result in transfer of at least 1% of the agent, at least 2% of the agent, at least 5% of the agent, at least 10% of the agent, at least 20% of the agent, at least 30% of the agent, or at least 40% of the agent.

The fluid may be a liquid in which the agent is dissolved, suspended, or otherwise uniformly distributed throughout the fluid. Preferably, the fluid is an aqueous liquid. The aqueous liquid may contain other components that stabilize the agent, such as salts, buffers, osmotic stabilizers, and the like.

The fluid should be a liquid conducive to transient acoustic cavitation. Generally, liquids with higher viscosity have a higher threshold for nucleation of bubbles and thus make transient cavitation more difficult. Consequently, the fluid may be a liquid with low viscosity. The liquid may have a viscosity that does not exceed a certain value. The liquid may have a dynamic viscosity that does not exceed a certain value. For example and without limitation, the liquid may have a dynamic viscosity that is not greater than about 0.25 mPa·s, not greater than about 0.5 mPa·s, not greater than about 0.75 mPa·s, not greater than about 1 mPa·s, not greater than about 1.25 mPa·s, or not greater than about 1.5 mPa·s. The liquid may have a kinematic viscosity that does not exceed a certain value. For example and without limitation, the liquid may have a kinematic viscosity that is not greater than about 0.25 cSt, not greater than about 0.5 cSt, not greater than about 0.75 cSt, not greater than about 1 cSt, not greater than about 1.25 cSt, or not greater than about 1.5 cSt.

The fluid may contain an excipient. The excipient may facilitate transfer of the agent or analysis or quantification of transfer of the agent. For example and without limitation, the excipient may be 1,2,4,5 benzenetetracarboxylic acid, 3,3′ thiodipropione acid, 8-arm poly(ethylene glycol), adipic acid, alpha-cyclodextrin, cysteine, didodecyl 3,3′-thiodipropionate, EDTA, fructose, glycerin, mannose, mucin, poloxamer 407, poly(lactide glycolide) acid, poly(vinyl alcohol), polyethoxylated castor oil, saccharin, sodium glycolate, sodium glycocholate, sodium taurodeoxycholate, or sodium thiosulfate.

The methods may include introducing via the esophagus fluid and/or an agent proximate, such as in contact with, an internal tissue. The fluid, the agent, or both may introduced prior to delivering the ultrasound energy, at the same time as delivering the ultrasound energy, or both before and during delivery of the ultrasound energy.

The methods may be used to deliver an antigen or immunotherapeutic agent to a type of mucosal tissue. The mucosal tissue may be gastrointestinal tissue, such as buccal tissue, gingival tissue, labial tissue, esophageal tissue, gastric tissue, intestinal tissue, colorectal tissue, or anal tissue. The mucosal tissue may be nasal tissue or vaginal tissue.

The methods may involve contacting the fluid with a wound, skin, or tissue being treated. Alternatively, the methods may involve non-contact ultrasound treatment. In non-contact ultrasound treatment, the fluid or liquid does not directly contact the wound, skin, or tissue being treated but is atomized and delivered as a spray. Non-contact ultrasound treatment of wounds generally is known in the art and described in, for example, Bell, A. L. and Cavorsi, J., Noncontact ultrasound therapy for adjunctive treatment of nonhealing wounds: retrospective analysis, Phys Ther. 2008 December; 88(12):1517-24. doi: 10.2522/ptj.20080009; Keltie, K., et al., Characterization of the ultrasound beam produced by the MIST therapy, wound healing system, Ultrasound Med Biol. 2013 July; 39(7):1233-40. doi: 10.1016/j.ultrasmedbio.2012.10.022; and Maan, Z. N. et al., Noncontact, Low-Frequency Ultrasound Therapy Enhances Neovascularization and Wound Healing in Diabetic Mice, Plast Reconstr Surg. 2014 September, 134(3): 402e-411e, doi: 10.1097/PRS.0000000000000467, the contents of each of which are incorporated herein by reference.

One or more of the steps described above may be repeated. For example, the methods may include repeating one or more of the introducing and delivering steps. The steps may be performed two, three, four, five, or more times. The steps may be repeated at defined intervals. For example and without limitation, the steps may be repeated at intervals of 5 minutes, 10 minutes, 15 minutes, 30 minutes, 1 hour, 2 hours, 4 hours, 4 hours, 1 day, 2 days, 3 days, 4 days, 5 days, 1 week, 2 weeks, 3 weeks, 4 weeks or more.

The methods may include repeated a method described above according to a schedule. The schedule may include repeated administrations of an agent to tissue at defined intervals for a defined period. For example, schedule may include repeated administration at intervals of about 1 day, about 2 days, about 3 days, about 4 days, about 5 days, or about 7 days over a period of about 1 week, about 2 weeks, about 3 weeks, about 4 weeks, about 6 weeks, about 8 weeks, or about 12 weeks.

The methods may include the use of a device of the invention, such as one of the devices described above in relation to devices of the invention.

The subject may be any type of subject, such as an animal, for example, a mammal, for example, a human. The subject may suffer from a disease, disorder or condition.

In certain embodiments of the invention, methods of ultrasound-based delivery of antigens trigger an immune response in the patient. Ultrasound stimulation of the fluid by itself triggers a response from immune cells. In addition, transient cavitation drives the antigen into the mucosal tissue, where immune cells mount a response to the antigen. Thus, the methods take advantage of the synergistic effects of ultrasound-based delivery to provide superior immunization than is possible with prior methods.

Due to the density of immune cells in mucosal tissue, the methods allow delivery of antigen directly to immune cells. The antigen may be delivered to a specific type of population of immune cells, such as B cells, basophils, eosinophils, lymphocytes, macrophages, mast cells, megakaryocytes, monocytes, myeloblasts, natural killer (NK) cells, neutrophils, T cells, T regulatory (T_{reg}) cells, naïve T cells, cytotoxic T cells, gamma delta T cells, natural killer T cells, effector T cells, helper T cells, CD25⁺ cells, CD4⁺ cells, CD3⁺ cells, or Foxp3⁺ cells. In addition, the density of immune cells residing in mucosal tissue may bias the immune response toward production of IgA antibodies rather than IgG antibodies.

### Agents for Ultrasound Delivery

The agent may be any agent that provides a therapeutic benefit. For example and without limitation, suitable agents include alpha-hydroxy formulations, ace inhibiting agents, analgesics, anesthetic agents, anthelmintics, anti-arrhythmic agents, antithrombotic agents, anti-allergic agents, anti-angiogenic agents, antibacterial agents, antibiotic agents, anticoagulant agents, anticancer agents, antidiabetic agents, anti-emetics, antifungal agents, antigens, antihypertension agents, antihypotensive agents, antiinflammatory agents, antimicotic agents, antimigraine agents, anti-obesity agents, antiparkinson agents, antirheumatic agents, antithrombins, antiviral agents, antidepressants, antiepileptics, antihistamines, antimuscarinic agents, antimycobacterial agents, antineoplastic agents, antithyroid agents, anxiolytics, asthma therapies, astringents, beta blocking agents, blood products and substitutes, bronchospamolytic agents, calcium antagonists, cardiovascular agents, cardiac glycosidic agents, carotenoids, cephalosporins, chronic bronchitis therapies, chronic obstructive pulmonary disease therapies, contraceptive agents, corticosteroids, cytostatic agents, cystic-fibrosis therapies, cardiac inotropic agents, contrast media, cough suppressants, diagnostic agents, diuretic agents, dopaminergics, elastase inhibitors, emphysema therapies, enkephalins, fibrinolytic agents, growth hormones, hemostatics, immunological agents, immunosupressants, immunotherapeutic agents, insulins, interferons, lactation inhibiting agents, lipid-lowering agents, lymphokines, muscle relaxants, neurologic agents, NSAIDS, nutraceuticals, oncology therapies, organ-transplant rejection therapies, parasympathomimetics, parathyroid calcitonin and biphosphonates, prostacyclins, prostaglandins, psycho-pharmaceutical agents, protease inhibitors, magnetic resonance diagnostic imaging agents, radio-pharmaceuticals, reproductive control hormones, respiratory distress syndrome therapies, sedative agents, sex hormones, somatostatins, steroid hormonal agents, stimulants and anoretics, sympathomimetics, thyroid agents, vasodilating agents, vitamins, and xanthines. A more complex list of chemicals and drugs that can be used as agents in embodiments of the invention is provided in the Merck Index: An Encyclopedia of Chemicals, Drugs, and Biologicals Fifteenth Edition, Maryadele J O'Neil, ed., RSC Publishing, 2015, ISBN-13: 978-1849736701, ISBN-10 1849736707, the contents of which are incorporated herein by reference.

Agents may be of any chemical form. For example, agents may be biological therapeutics, such as nucleic acids, proteins, peptides, polypeptides, antibodies, or other macromolecules. Nucleic acids include RNA, DNA, RNA/DNA hybrids, and nucleic acid derivatives that include non-naturally-occurring nucleotides, modified nucleotides, non-naturally-occurring chemical linkages, and the like. Examples of nucleic acid derivatives and modified nucleotides are described in, for example, International Publication WO 2018/118587, the contents of which are incorporated herein by reference. Nucleic acids may be polypeptide-encoding nucleic acids, such as mRNAs and cDNAs. Nucleic acids may interfere with gene expression. Examples of interfering RNAs (RNAi) include siRNAs and miRNAs. RNAi is known in the art and described in, for example, Kim and Rossi, Biotechniques. 2008 April; 44(5): 613-616, doi: 10.2144/000112792; and Wilson and Doudna, Molecular Mechanisms of RNA Interference, Annual Review of Biophysics 2013 42:1, 217-239, the contents of each of which are incorporated herein by reference. Agents may be organic molecules of non-biological origin. Such drugs are often called small-molecule drugs because they typically have a molecular weight of less than 2000 Daltons, although they may be larger. Agents may be combinations or complexes of one or more biological macromolecules and/or one or more small molecules. For example and without limitation, agents may be nucleic acid complexes, protein complexes, protein-nucleic acid complexes, and the like. Thus, the agent may exist in a multimeric or polymeric form, including homocomplexes and heterocomplexes.

The agent may be unformulated, i.e., it may be provided in a biologically active format that does not contain other molecules that interact with the agent solely to facilitate delivery of the agent. Formulations commonly used for delivery of biologic and small-molecule agents include viral particles, viral capsids, liposomes, vesicles, micelles, and complexes with other macromolecules that are not essential for the biological or biochemical function of the agent. Thus, the agent may be provided in a non-encapsulated form or in a form that is not complexed with other molecules unrelated to the function of the agent.

The agent may be a component of a gene editing system, such as a meganuclease, zinc finger nuclease (ZFN), a transcription activator-like effector-based nuclease (TALEN), or the clustered, regularly-interspersed palindromic repeat (CRISPR) system.

Meganucleases are endodeoxyribonucleases that recognize double-stranded DNA sequences of 12-40 base pairs. They can be engineered to bind to different recognition sequences to create customized nucleases that target particular sequences. Meganucleases exist in archaebacterial, bacteria, phages, fungi, algae, and plants, and meganucleases from any source may be used. Engineering meganucleases to recognize specific sequences is known in the art and described in, for example, Stoddard, Barry L. (2006) “Homing endonuclease structure and function” Quarterly Reviews of Biophysics 38 (1): 49-95 doi:10.1017/S0033583505004063, PMID 16336743; Grizot, S.; Epinat, J. C.; Thomas, S.; Duclert, A.; Rolland, S.; Paques, F.; Duchateau, P. (2009) “Generation of redesigned homing endonucleases comprising DNA-binding domains derived from two different scaffolds” Nucleic Acids Research 38 (6): 2006-18, doi:10.1093/nar/gkp1171. PMC 2847234, PMID 20026587; Epinat, Jean-Charles; Arnould, Sylvain; Chames, Patrick; Rochaix, Pascal; Desfontaines, Dominique; Puzin, Clémence; Patin, Amélie; Zanghellini, Alexandre; Pâques, Frédéric (Jun. 1, 2003) “A novel engineered meganuclease induces homologous recombination in yeast and mammalian cells” Nucleic Acids Research 31 (11): 2952-2962; and Seligman, L. M.; Chisholm, K M; Chevalier, B S; Chadsey, M S; Edwards, S T; Savage, J H; Veillet, A L (2002) “Mutations altering the cleavage specificity of a homing endonuclease” Nucleic Acids Research 30 (17): 3870-9, doi:10.1093/nar/gkf495. PMC 137417, PMID 12202772, the contents of each of which are incorporated herein by reference.

ZFNs are artificial restriction enzymes that have a zinc finger DNA-binding domain fused to a DNA-cleavage domain. ZFNs can also be engineered to target specific DNA sequences. The design and use of ZFNs is known in the art and described in, for example, Carroll, D (2011) “Genome engineering with zinc-finger nucleases” Genetics Society of America 188 (4): 773-782, doi:10.1534/genetics.111.131433. PMC 3176093, PMID 21828278; Cathomen T, Joung J K (July 2008) “Zinc-finger nucleases: the next generation emerges” Mol. Ther. 16 (7): 1200-7, doi:10.1038/mt.2008.114, PMID 18545224; Miller, J. C.; Holmes, M. C.; Wang, J.; Guschin, D. Y.; Lee, Y. L.; Rupniewski, I.; Beausejour, C. M.; Waite, A. J.; Wang, N. S.; Kim, K. A.; Gregory, P. D.; Pabo, C. O.; Rebar, E. J. (2007) “An improved zinc-finger nuclease architecture for highly specific genome editing” Nature Biotechnology, 25 (7): 778-785, doi:10.1038/nbt1319, PMID 17603475, the contents of each of which are incorporated herein by reference.

TALENs are artificial restriction enzymes that have a TAL effector DNA-binding domain fused to a DNA cleavage domain. TALENs can also be engineered to target specific DNA sequences. The design and use of TALENs is known in the art and described in, for example, Boch J (February 2011) “TALEs of genome targeting” Nature Biotechnology 29 (2): 135-6, doi:10.1038/nbt.1767. PMID 21301438; Juillerat A, Pessereau C, Dubois G, Guyot V, Maréchal A, Valton J, Daboussi F, Poirot L, Duclert A, Duchateau P (January 2015) “Optimized tuning of TALEN specificity using non-conventional RVDs” Scientific Reports, 5: 8150, doi:10.1038/srep08150. PMC 4311247, PMID 25632877; and Mahfouz M M, Li L, Shamimuzzaman M, Wibowo A, Fang X, Zhu J K (February 2011) “De novo-engineered transcription activator-like effector (TALE) hybrid nuclease with novel DNA binding specificity creates double-strand breaks” Proceedings of the National Academy of Sciences of the United States of America, 108 (6): 2623-8, Bibcode:2011PNAS,108.2623M, doi:10.1073/pnas.1019533108, PMC 3038751, PMID 21262818, the contents of each of which are incorporated herein by reference.

The CRISPR system is a prokaryotic immune system that provides acquired immunity against foreign genetic elements, such as plasmids and phages. CRISPR systems include one or more CRISPR-associated (Cas) proteins that cleave DNA at clustered, regularly-interspersed palindromic repeat (CRISPR) sequences. Cas proteins include helicase and exonuclease activities, and these activities may be on the same polypeptide or on separate polypeptides. Cas proteins are directed to CRISPR sequences by RNA molecules. A CRISPR RNA (crRNA) binds to a complementary sequence in the target DNA to be cleaved. A transactivating crRNA (tracrRNA) binds to both the Cas protein and the crRNA to draw the Cas protein to the target DNA sequence. Not all CRISPR systems require tracrRNA. In nature crRNA and tracrRNA occur on separate RNA molecules, but they also function when contained a single RNA molecule, called a single guide RNA or guide RNA (gRNA). The one or more RNAs and one or more polypeptides assemble inside the cell to form a ribonucleoprotein (RNP). CRISPR systems are described, for example, in van der Oost, et al., CRISPR-based adaptive and heritable immunity in prokaryotes, Trends in Biochemical Sciences, 34(8):401-407 (2014); Garrett, et al., Archaeal CRISPR-based immune systems: exchangeable functional modules, Trends in Microbiol. 19(11):549-556 (2011); Makarova, et al., Evolution and classification of the CRISPR-Cas systems, Nat. Rev. Microbiol. 9:467-477 (2011); and Sorek, et al., CRISPR-Mediated Adaptive Immune Systems in Bacteria and Archaea, Ann. Rev. Biochem. 82:237-266 (2013), the contents of each of which are incorporated herein by reference.

CRISPR-Cas systems have been placed in two classes. Class 1 systems use multiple Cas proteins to degrade nucleic acids, while class 2 systems use a single large Cas protein. Class 1 Cas proteins include Cas10, Cas10d, Cas3, Cas5, Cas8a, Cmr5, Cse1, Cse2, Csf1, Csm2, Csx11, Csy1, Csy2, and Csy3. Class 2 Cas proteins include C2c1, C2c2, C2c3, Cas4, Cas9, Cpf1, and Csn2.

CRISPR-Cas systems are powerful tools because they allow gene editing of specific nucleic acid sequences using a common protein enzyme. By designing a guide RNA complementary to a target sequence, a Cas protein can be directed to cleave that target sequence. In addition, although naturally-occurring Cas proteins have endonuclease activity, Cas proteins have been engineered to perform other functions. For example, endonuclease-deactivated mutants of Cas9 (dCas9) have been created, and such mutants can be directed to bind to target DNA sequences without cleaving them. dCas9 proteins can then be further engineered to bind transcriptional activators or inhibitors. As a result, guide sequences can be used to recruit such CRISPR complexes to specific genes to turn on or off transcription. Thus, these systems are called CRISPR activators (CRISPRa) or CRISPR inhibitors (CRISPRi). CRISPR systems can also be used to introduce sequence-specific epigenetic modifications of DNA, such acetylation or methylation. The use of modified CRISPR systems for purposes other than cleavage of target DNA are described, for example, in Dominguez, et al., Beyond editing: repurposing CRISPR-Cas9 for precision genome regulation and interrogation, Nat. Rev. Cell Biol. 17(1):5-15 (2016), which is incorporated herein by reference.

The agent may be any component of a CRISPR system, such as those described above. For example and without limitation, the CRISPR component may be one or more of a helicase, endonuclease, transcriptional activator, transcriptional inhibitor, DNA modifier, gRNA, crRNA, or tracrRNA. The CRISPR component contain a nucleic acid, such as RNA or DNA, a polypeptide, or a combination, such as a RNP. The CRISPR nucleic acid may encode a functional CRISPR component. For example, the nucleic acid may be a DNA or mRNA. The CRISPR nucleic acid may itself be a functional component, such as a gRNA, crRNA, or tracrRNA.

The agent may include an element that induces expression of the CRISPR component. For example, expression of the CRISPR component may be induced by an antibiotic, such as tetracycline, or other chemical. Inducible CRISPR systems have been described, for example, in Rose, et al., Rapidly inducible Cas9 and DSB-ddPCR to probe editing kinetics, Nat. Methods, 14, pages 891-896 (2017); and Cao, et al., An easy and efficient inducible CRISPR/Cas9 platform with improved specificity for multiple gene targeting, Nucleic Acids Res. 14(19):e149 (2016), the contents of which are incorporated herein by reference. The inducible element may be part of the CRISPR component, or it may be a separate component.

The agent may have a minimum size. For example and without limitation, the agent may have a molecular weight of >100 Da, >200 Da, >500 Da, >1000 Da, >2000 Da, >5000 Da, >10,000 Da, >20,000 Da, >50,000 Da, or >100,000 Da.

Ultrasound-based immunization methods of the invention may be used to deliver an antigen. The antigen may be any molecule, complex of molecules, or portion of pathogen that elicits an immune response from the host. For example and without limitation, the antigen may be or may contain a nucleic acid, a peptide, a polypeptide, a protein, an antibody, an organic molecule, a carbohydrate, a polysaccharide, a lipid, a toxoid, or any combination thereof. The nucleic acid may be RNA, DNA, or a RNA/DNA hybrid. The nucleic acid may be single-stranded or double-stranded.

Toxoids are derivatives of toxins that have been modified to inactivate or suppress their toxicity while retaining their immunogenicity. The source of the toxin is a bacterium or other pathogen. Toxoids are typically produced by heating or chemically modifying the toxin. Complete detoxification of a toxin can be achieved more easily and reliably than inactivation of a pathogenic bacterium or virus, so toxoid vaccines are generally safer than live-attenuated vaccines. In addition, because toxoids are simpler structurally, they are less prone than live-attenuated vaccines to degradation and loss of immunogenicity and therefore easier to store and transport. Common toxoid vaccines include toxoids derived from tetanospasmin, botulin, and pertussis toxin.

The antigen may be or may include a live-attenuated pathogen, an inactivated or killed pathogen, or a subunit or conjugate of pathogen. Live-attenuated vaccines are produced by eliminating or reducing the virulence of a pathogen without killing or completely inactivating it. Live-attenuated vaccines are widely used to immunize patients against influenza, measles, mumps, polio, rotavirus, rubella, smallpox, tuberculosis, typhoid, typhus, varicella (chicken pox), and yellow fever. Inactivated/killed vaccines contain pathogen particles that have been killed or inactivated. Inactivated/killed vaccines are used to immunize patients against cholera, influenza, pertussis (whooping cough), the plague (Yersinia), polio, and typhoid. Subunit vaccines present individual proteins from a pathogen to the patient either in isolation or exogenously expressed in a different organism. Subunit vaccines are used to immunize patients against hepatitis B, influenza, haemophilus influenza type b (Hib), human immunodeficiency virus (HIV), human papilloma virus (HPV), pertussis (whooping cough) pneumococcal disease, meningococcal disease, and shingles.

An advantage of ultrasound-based delivery of agents, including antigens and immunotherapeutic agents, to mucosal tissue is the capacity to deliver large molecules, e.g., molecules having a molecular weight greater than 1000 Da. Thus, the antigen may have a minimum size. For example and without limitation, the antigen may have a molecular weight of >100 Da, >200 Da, >500 Da, >1000 Da, >2000 Da, >5000 Da, >10,000 Da, >20,000 Da, >50,000 Da, or >100,000 Da.

The antigen may be derived from a particular pathogen. The pathogen may be a bacterium, virus, fungus, or protozoa. For example and without limitation, the pathogen may be Bordetella pertussis, Brugia malayi, Brugia timori, Clostridium difficile, Cryptosporidium hominis, Cryptosporidium parvum, Haemophilus influenzae, a hepatitis virus, human papilloma virus, influenza virus, measles virus, mumps virus, Neisseria meningitidis (meningococcus), Orientia tsutsugamushi, poliovirus, Rickettsia prowazekii, Rickettsia typhi, rotavirus, rubella virus, Salmonella enterica, Streptococcus pneumoniae, Trypanosoma brucei, Trypanosoma cruzi, varicella zoster virus, Variola major, Variola minor, Vibrio cholerae, Wuchereria bancrofti, yellow fever virus, or Yersinia testis.

The antigen may provide immunity or resistance to any infectious disease. For example and without limitation, the infectious disease may be Chagas, cholera, diarrhea, hepatitis, HIV, HPV, human African trypanosomiasis, influenza, lymphatic filariasis, measles, meningococcal disease, mumps, pertussis (whooping cough), plague, pneumococcal disease, polio, rubella, shingles, smallpox, typhoid, typhus, or yellow fever.

The antigen may be provided in a formulation that improves delivery or antigenicity of the antigen. For example, the antigen may be provided as a free antigen molecule that is not encapsulated or contained in macromolecular complex. Because ultrasound-based delivery allows rapid delivery to target cells without requiring the antigen to enter systemic circulation, such formats are not required for stabilization or protection of the antigen. Alternatively or additionally, the antigen may be provided in format that allows for sustained and prolonged release of the antigen into the mucosal tissue or from the mucosal tissue into circulation.

The antigen may be provided in a formulation that prolongs release of the antigen, such as a depot system. For example, the depot system may delay exposure of the antigen to cells or the circulatory system and/or delay clearance of the antigen from the body. A variety of extended-release formulations are known in the art. For example and without limitation, the formulation may include archaeosomes, colloidal iron-based preparations, dendrimers, E2 multimeric scaffolds, emulsions, gels, hematin anhydride crystals, hydrogel capsules, immune stimulating complexes (ISCOMs), lipid vesicles, liposomes, LPD (liposomes-protamine-DNA complexes), micromolded polymers, microneedles, microparticles, nanoparticles, niosomes, PEGylated liposomes, polymerized targeted-liposomes, solid lipid nanoparticles (SLNs), three-dimensional printed polymers, virosomes, or virus-like particles (VLPs). The formulation may be degradable, bioerodible, or non-degradable. The formulation may include a polymer or specific chemical component. For example and without limitation, the formulation may contain chitosan, gamma polyglutamic acid (γ-PGA), gelatin, hematin anhydride, hyaluronan, hyaluronic acid, latex, poly-(1,4-phenyleneacetone dimethylene thioketal), poly(alkylcyanoacrylate) (PACA), poly(lactic-co-glycolic acid) (PLGA), poly(methyl methacrylate) (PMMA), poly(phosphazenes), poly-alkyl-cyano-acrylates (PAC), polyanhydrides, polylactic acid (PLA), or poly-ε-caprolactone (PCL). Formulations for extended release of antigens are described in, for example, Chen, M. C., et al., Enhancing immunogenicity of antigens through sustained intradermal delivery using chitosan microneedles with a patch-dissolvable design, Acta Biomater. 2018 January; 65:66-75. doi: 10.1016/j.actbio.2017.11.004; D. S. Wilson, et al., “Orally delivered thioketal nanoparticles loaded with TNF-α-siRNA target inflammation and inhibit gene expression in the intestines,” Nature Materials, vol. 9, no. 11, pp. 923-928, October 2010; Ishii-Mizuon, Y. et al., Improved sustained release of antigen from immunostimulatory DNA hydrogel by electrostatic interaction with chitosan, Int J Pharm. 2017 Jan. 10; 516(1-2):392-400. doi: 10.1016/j.ijpharm.2016.11.048; Trovato, M. and De Berardinis, P., Novel antigen delivery systems, World J Virol 2015 Aug. 12; 4(3): 156-168, ISSN 2220-3249, DOI: 10.5501/wjv.v4.i3.156; International Application Nos. WO 2000/041682 and WO 2001/039800; U.S. Pat. Nos. 8,173,104 and 8,974,795; and U.S. Publication No. 2015/0165020, the contents of each of which are incorporated herein by reference.

The formulation may contain an adjuvant that stimulates the immune response to the antigen. Many adjuvants are known in the art. For example and without limitation, the adjuvant may be or may contain aluminum sulfate, aluminum hydroxide, aluminum phosphate, calcium phosphate hydroxide, paraffin oil, peanut oil, killed bacteria, such as Bordetella pertussis or Mycobacterium bovis, toxoids, squalene, detergents, plant saponins from Quillaia, soybean, or Polygala senega, cytokines, such as IL-1, IL-2, or IL-12, Freund's complete adjuvant, and Freund's incomplete adjuvant. Adjuvants are known in the art and described in, for example, Greenfield, E., ed., Antibodies: A Laboratory Manual, Second edition, Cold Spring Harbor Laboratory Press, Cold Spring Harbor, N.Y. (2014), ISBN 978-1-936113-81-1; Lowrie D. B. and Whalen R. G., DNA Vaccines: Methods and Protocols. Humana Press, ISBN 978-0-89603-580-5; and U.S. Publication No. 2017/0209568, the contents of each of which are incorporated herein by reference.

Alternatively, the formulation may be free of chemical or biological adjuvants. The ultrasound energy itself stimulates the response of immune cells, as described below. Therefore, ultrasound-based delivery of the antigen may produce a sufficient immune reaction to the antigen, and the methods may obviate the need for more complicated, adjuvant-based formulations.

The formulation may contain a marker that can be used to evaluate the efficiency of delivery of the antigen to the mucosal tissue. Any marker that provides a measurable signal can be used. The signal may a visual signal. For example, the marker may be fluorescent, luminescent, or phosphorescent.

In certain embodiments of the invention, methods include ultrasound-based delivery of immunotherapeutic agents to mucosal tissue. The principles of the process are the same as described above in relation to antigen delivery, but the cargo includes agents that provide therapeutic benefit. Immunotherapeutic agents include agents derived from or based on immunoglobulin molecules, such as antibodies. An antibody may be a full-length antibody, a fragment of an antibody, a naturally occurring antibody, a synthetic antibody, an engineered antibody, a full-length affibody, a fragment of an affibody, a full-length affilin, a fragment of an affilin, a full-length anticalin, a fragment of an anticalin, a full-length avimer, a fragment of an avimer, a full-length DARPin, a fragment of a DARPin, a full-length fynomer, a fragment of a fynomer, a full-length kunitz domain peptide, a fragment of a kunitz domain peptide, a full-length monobody, a fragment of a monobody, a peptide, a polyaminoacid, or the like.

Immunotherapeutic agents also include immunomodulators. Immunomodulators stimulate or regulate activity of the immune system. For example and without limitation, immunomodulators include interleukins, cytokines, chemokines, immunomodulatory imide drugs, cytosine phosphate-guanosine, oligodeoxynucleotides, and glucans. The interleukin may be IL-2, IL-7, or IL-12. The cytokine may be an interferon or G-CSF. The chemokine may be CCL3, CCL26, or CXCL7. The immunomodulatory imide drug may be thalidomide, lenalidomide, pomalidomide, or apremilast.

Other immunotherapeutic agents include antimicrobials, antibiotics, antivirals, and antifungals.

In certain embodiments of the invention, methods allow delivery of agents that promote wound healing. The agent may promote healing by any mechanism. For example and without limitation, the agent may facilitate one or more phases of the wound healing process, as described above; prevent infection, including bacterial or viral infection; or alleviate pain or sensitivity. Preferably, the agent is a growth factor.

A variety of growth factors promote wound healing. For example and without limitation, growth factors that promote wound healing include CTGF/CCN2, EGF family members, FGF family members, G-CSF, GM-CSF, HGF, HGH, HIF, histatin, hyaluronan, IGF, IL-1, IL-4, IL-8, KGF, lactoferrin, lysophosphatidic acid, NGF, a PDGF, TGF-β, and VEGF. The EFG family includes 10 members: amphiregulin (AR), betacellulin (BTC), epigen, epiregulin (EPR), heparin-binding EGF-like growth factor (HB-EGF), neuregulin-1 (NRG1), neuregulin-2 (NRG2), neuregulin-3 (NRG3), neuregulin-4 (NRG4), or transforming growth factor-α (TGF-α). The FGF family includes 22 members: FGF1, FGF2 (also called basic FGF or bFGF), FGF3, FGF4, FGF5, FGF6, FGF7, FGF8, FGF9, FGF10, FGF11, FGF12, FGF13, FGF14, FGF16, FGF17, FGF18, FGF19, FGF20, FGF21, FGF22, or FGF23. PDGF exists in three forms: PDGF AA, PDGF AB, and PDGF BB. The TGF-β family includes three forms: TGF-β1, TGF-β2, and TGF-β3.

A variety of agents that prevent infection have been used to treat wounds. For example and without limitation, the agent may be an antimicrobial, antiviral, antibiotic, antifungal, or antiseptic. Exemplary agents include silver, iodine, chlorhexidine, hydrogen peroxide, lysozyme, peroxidase, defensins, cystatins, thrombospondin, and antibodies. Nitric oxide donors, such as glyceryl trinitrate and nitrite salts, are also useful to prevent infection and promote wound healing.

### Wounds, Including Diabetic Ulcers

In certain embodiments, methods of the invention are useful for treating wounds. For example, the wound may be a burn or an ulcer, such as from diabetes, a surgical incision or stitching, skin graft, hair transplant, bed sore, tissue dehiscence, or ligament or tendon repair. The wound may be on the skin, or it may be on a mucosal membrane. For example, the wound may be a mouth ulcer, canker sore, peptic ulcer, gastric ulcer, duodenal ulcer, or corneal ulcer.

Wound healing occurs in a series of phases. In the first phase, called hemostasis or blood clotting, platelets form a clot that prevents further bleeding. Next, damaged cells, pathogens, and debris are removed from the wound site during the inflammation phase. Platelet-derived growth factors (PDGF) released into the wound promote the next phase, proliferation. Proliferation involves angiogenesis, collagen deposition, granulation tissue formation, epithelialization, and wound contraction. Angiogenesis entails the formation of new blood vessels by vascular endothelial cells in response to vascular endothelial growth factor. The growth and migration of fibroblasts in response to fibroblast growth factors 1 and 2 (FGF1 and FGF2), PDGF, and tumor growth factor-β (TGF-β) leads to collagen deposition and formation of granulation tissue. Epithelialization occurs by the movement and proliferation of keratinocytes along the wound bed in response to epidermal growth factors (EGFs), keratinocyte growth factor (KGF), tumor growth factor-α (TGF-α), and insulin-like growth factor (IGF). Wound contraction is mediated by the pulling action of myofibroblasts in response to various growth factors. In maturation, the final phase of wound healing, collagen is realigned along tension lines. Wound healing is described in detail in, for example, Clark, R. A. F., “Overview and General Consideration of Wound Repair. The Molecular and Cellular Biology of Wound Repair.” Plenum Press. New York (1996)3-50; Annu. Rev. Med. 46:467-481(1995); J. Pathol. 178: 5-10(1996); Nguyen, D. T., et al., “4 The Pathophysiologic Basis for Wound Healing and Cutaneous Regeneration” Biomaterials for Treating Skin Loss, Elsevier, pp. 25-57, Orgill D. P., Blanco C. (editors), ISBN 978-1-84569-554-5; and Stadelmann, W. K.; et al., “Physiology and healing dynamics of chronic cutaneous wounds” American journal of surgery, 176 (2A Suppl): 26S-38S, doi:10.1016/S0002-9610(98)00183-4, PMID 9777970, the contents of each of which are incorporated herein by reference.

Diabetes and other disorders can impair the wound healing process. In diabetes, formation of advanced glycation endproducts (AGEs), proteins in which sugar molecules are non-enzymatically attached to the polypeptide at random, impairs the function of proteins of the extracellular matrix, thereby disrupting granulation tissue formation. AGE accumulation is also a factor in progression of degenerative diseases such as atherosclerosis, chronic kidney disease, and Alzheimer's disease, and chronic kidney disease is also associated with poor wound healing. Diabetics may also display reduce ability to generate nitric oxide, which regulate angiogenesis. In addition, fibroblasts from diabetic patients are morphologically and functionally abnormal. Finally, diabetic patients often display elevated activity of matrix metalloproteinases (MMPs), which break down components of the extracellular matrix. High levels of MMPs interfere with construction of the extracellular matrix required to guide migration of fibroblasts and keratinocytes and therefore impede the proliferation phase of wound healing.

Certain factors increase the risk of development of diabetic ulcers. For example, infection, older age, diabetic neuropathy, peripheral vascular disease, cigarette smoking, poor glycemic control, previous foot ulcerations or amputations, ischemia of small and large blood vessels, prior history of foot disease, foot deformities that produce abnormally high forces of pressure, renal failure, edema, impaired ability to look after personal care (e.g. visual impairment) are all risk factors for development of diabetic ulcers.

### Specific Embodiments of the Invention

In certain aspects, the invention provides a method for immunizing a subject, the method comprising introducing a fluid comprising an antigen proximate a mucosal tissue of a subject and delivering ultrasound energy to the fluid at a frequency that causes the antigen to enter the mucosal tissue of the subject, thereby initiating an immune response that results in immunization of the subject.

In some embodiments of the method, the introducing step and the delivering step are not repeated. In some embodiments of the method, the introducing step and the delivering step are repeated.

The mucosal tissue may be gastrointestinal tissue. The gastrointestinal tissue may be buccal tissue.

The fluid may include a formulation that prolongs release of the antigen into the mucosal tissue.

The antigen may be a nucleic acid, a peptide, a polypeptide, a protein, an antibody, an organic molecule, a toxoid, or any combination thereof.

The antigen may have a molecular weight of >1000 Da.

The immune response may include activation of effector T cells, such may be CD25⁺ cells. The immune response may include recruitment of immune cells, dendritic cells, or both to a site at which the antigen entered the mucosal tissue.

Delivering the ultrasound energy may produce transient cavitation of the fluid. Implosion of bubbles in the fluid may propel the antigen into the mucosal tissue.

In certain aspects, the invention provides a method for immunizing a subject, the method comprising introducing a fluid comprising an antigen proximate a mucosal tissue of a subject and delivering ultrasound energy to the fluid at a frequency that results in an immune response and causes the antigen to enter the mucosal tissue of the subject, which also results in the immune response, wherein the combination results in immunization of the subject.

In some embodiments of the method, the introducing step and the delivering step are not repeated. In some embodiments of the method, the introducing step and the delivering step are repeated.

The mucosal tissue may be gastrointestinal tissue. The gastrointestinal tissue may be buccal tissue.

The fluid may include a formulation that prolongs release of the antigen into the mucosal tissue.

The antigen may be a nucleic acid, a peptide, a polypeptide, a protein, an antibody, an organic molecule, a toxoid, or any combination thereof.

The antigen may have a molecular weight of >1000 Da.

The immune response may include activation of effector T cells, such may be CD25⁺ cells. The immune response may include recruitment of immune cells, dendritic cells, or both to a site at which the antigen entered the mucosal tissue.

Delivering the ultrasound energy may produce transient cavitation of the fluid. Implosion of bubbles in the fluid may propel the antigen into the mucosal tissue.

In certain aspects, the invention provides a method for treating a target tissue of a subject, the method comprising delivering ultrasound energy to a subject at a frequency that initiates an immune response directed at a target tissue of the subject and delivering ultrasound energy to a fluid at a frequency that causes an immunotherapeutic agent in the fluid to enter the target tissue of the subject, wherein a combination of the immune response and the immunotherapeutic agent provides a treatment to the target tissue of the subject.

The target tissue may be mucosal tissue.

The immunotherapeutic agent may be an antibody, an antimicrobial, a chemokine, a cytokine, an imide drug, or an interleukin.

The ultrasound energy that causes the immunotherapeutic agent in the fluid to enter the target tissue may be delivered at a frequency of from about 20 kHz to about 60 kHz. The ultrasound energy may produce transient cavitation of the fluid.

Implosion of bubbles in the fluid may propels the immunotherapeutic agent into the target tissue.

The immunotherapeutic agent may be propelled into immune cells.

In certain aspects, the invention provides a method of treating a wound in a subject, the method comprising providing a fluid comprising an agent that promotes wound healing and delivering ultrasound energy to the fluid at a frequency to produce transient cavitation of the fluid to propel the agent into the wound of a subject, thereby treating the wound in the subject.

The agent may be an analgesic, an antibiotic, an anticoagulant, an antimicrobial, an antioxidant, an antiseptic, a calcium channel blocker, a corticosteroid, a growth factor, honey, a methylxanthine, a nitric oxide donor, phenytoin, a prostacyclin analog, a retinoid, or a nucleic acid encoding any of the aforementioned agents. The growth factor may be EGF, FGF1, FGF2, HGF, KGF TGF-α, TGF-β, or VEGF.

The wound may be an abrasion, a bedsore, a burn, a cosmetic blemish, a decubitus ulcer, a laceration, pressure gangrene, a surgical incision, or an ulcer.

The ultrasound energy may be delivered at a frequency of from about 20 kHz to about 60 kHz. The ultrasound energy may be delivered in a pulse of from about 10 seconds to about 3 minutes. The ultrasound energy may result in breakdown of less than about 50% of the agent. The ultrasound energy may be delivered from an ultrasound device comprising a horn in contact with the fluid.

In certain aspects, the invention provides a method of treating a diabetic ulcer in a subject, the method comprising providing a fluid comprising an agent that promotes healing of a diabetic ulcer and delivering ultrasound energy to the fluid at a frequency to produce transient cavitation of the fluid to propel the agent into the diabetic ulcer of a subject, thereby treating the diabetic ulcer in the subject.

The agent may be an analgesic, an antibiotic, an anticoagulant, an antimicrobial, an antioxidant, an antiseptic, a calcium channel blocker, a corticosteroid, a growth factor, honey, a methylxanthine, a nitric oxide donor, phenytoin, a prostacyclin analog, a retinoid, or any combination thereof. The growth factor may be EGF, FGF1, FGF2, HGF, KGF TGF-α, TGF-β, or VEGF.

The subject may have a condition, such as cigarette smoking, diabetic neuropathy, edema, elderly status, a foot deformity, an infection, ischemia, limb amputation, peripheral vascular disease, poor glycemic control, or renal failure.

The ultrasound energy may be delivered at a frequency of from about 20 kHz to about 60 kHz. The ultrasound energy may be delivered in a pulse of from about 10 seconds to about 3 minutes. The ultrasound energy may results in breakdown of less than about 50% of the agent. The ultrasound energy is delivered from an ultrasound device comprising a horn in contact with the fluid.

The device may include a chamber that holds at least a portion of the fluid.

### EXAMPLES

### Example 1

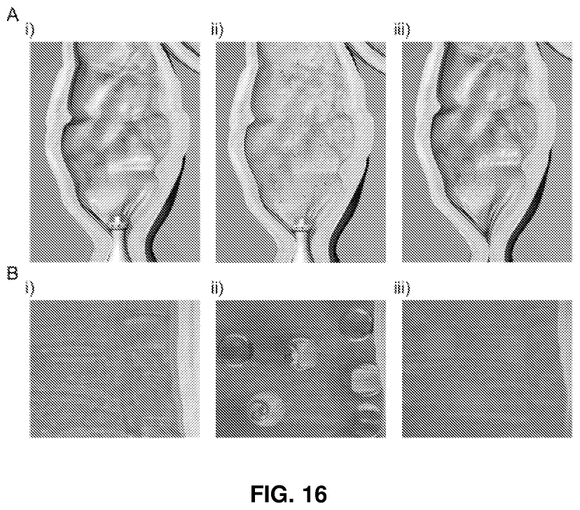

FIG. 16 shows macroscopic (A) and microscopic (B) images of the use of ultrasound in the colon for the ultra-rapid delivery of material to a mucosal surface. i) Treatment is started by inserting the enema syringe into the colon. ii) When treatment is started, the material to be delivered is instilled in the colon and low frequency ultrasound nucleates cavitation bubbles, which implode and drive microjets of drug (light blue) into the tissue. iii) After treatment, the device is removed and the depot systems preferentially reside locally in the tissue to achieve extended release of antigen.

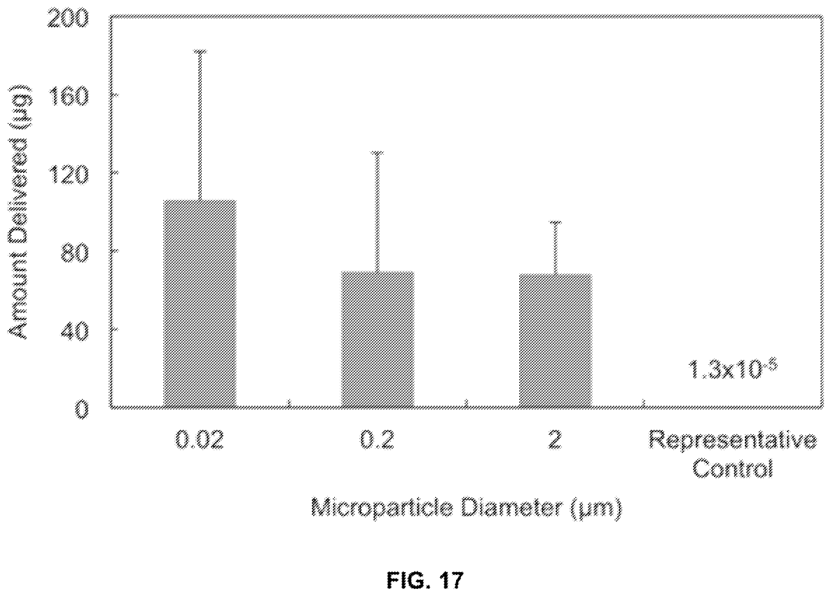

FIG. 17 is a graph showing delivery of fluorescently labeled latex particles of varying diameters into porcine colonic tissue ex vivo. Data represent averages+one standard deviation.

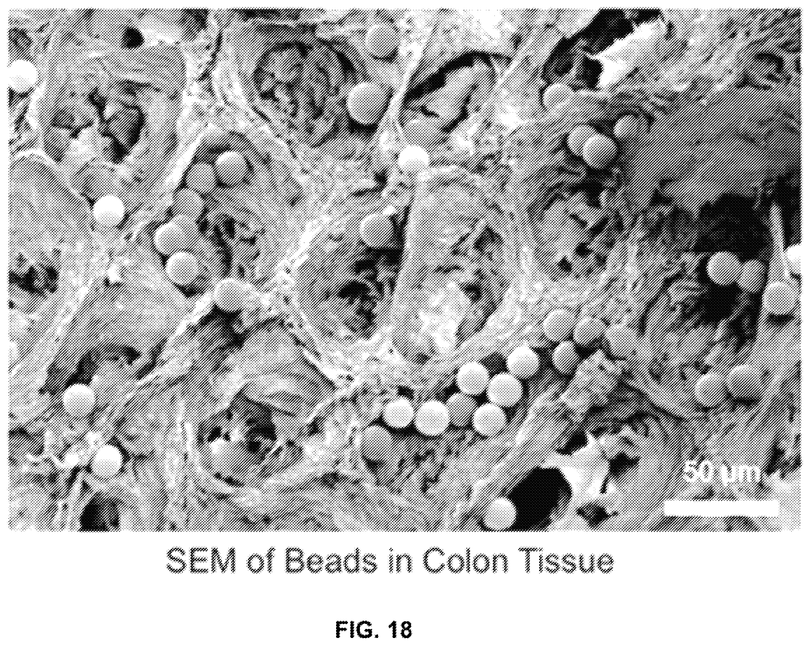

FIG. 18 is a Scanning Electron Microscopy (SEM) image of latex beads evenly disbursed over colonic tissue immediately after delivery with ultrasound.

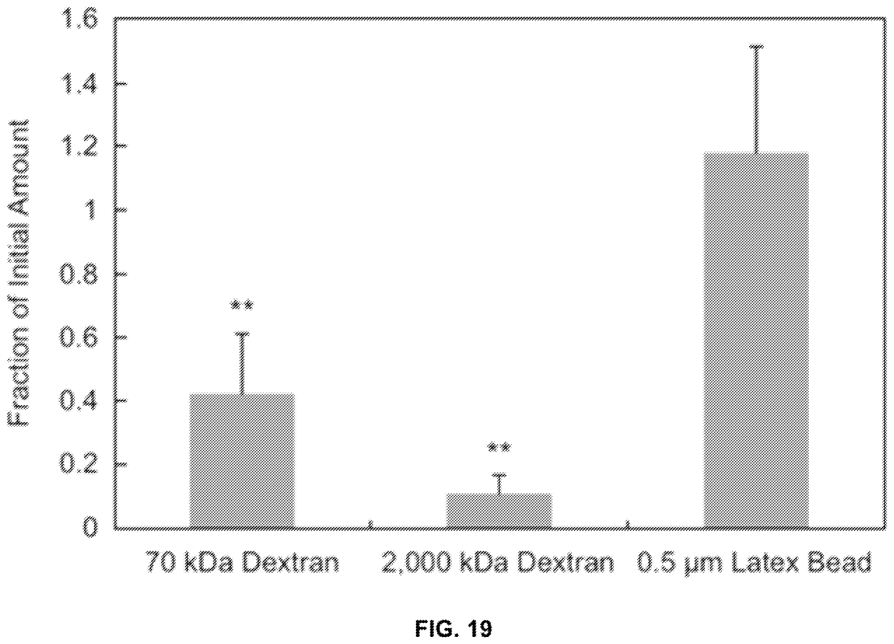

FIG. 19 is a graph showing the fraction of the initial amount of material delivered into colonic tissue 24 hours after delivery for various fluorescently labeled permeants. Data represents averages+one standard deviation. ** represents P<0.05 compared to the amount of each permeant delivered into tissue immediately after treatment by a two-tailed Student's t-test.

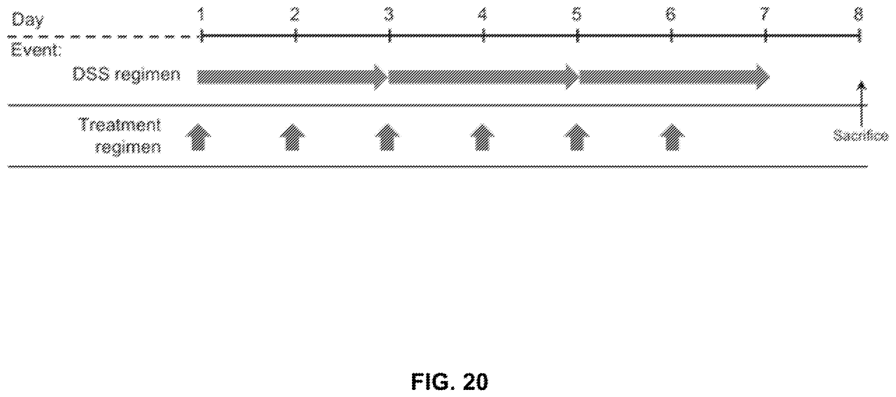

FIG. 20 is a schematic showing induction of dextran sulfate sodium-induced colitis and enema administration schedule. Dextran sulfate sodium was given for 7 consecutive days to induce acute colitis in mice. Concurrently, animals were administered enemas from day 1 through 6. Experimental groups consisted of either 200 ng of siRNA targeting Tnf mRNA in combination with ultrasound or 200 ng of siRNA targeting Tnf mRNA alone.

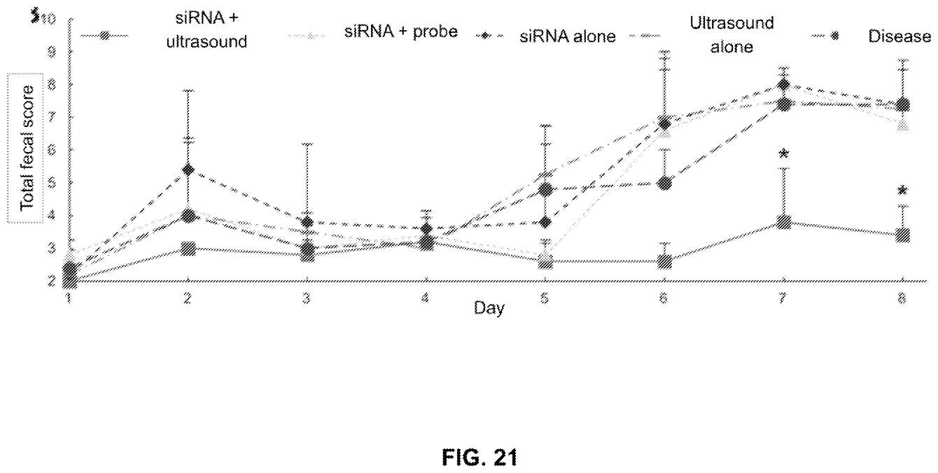

FIG. 21 is a graph showing total fecal score for animals with acute colitis receiving various enemas (n=5 animals per group). * represents P<0.021 for siRNA+ultrasound compared to all other groups (determined by one-way ANOVA with multiple comparisons). Data represent averages+one standard deviation.

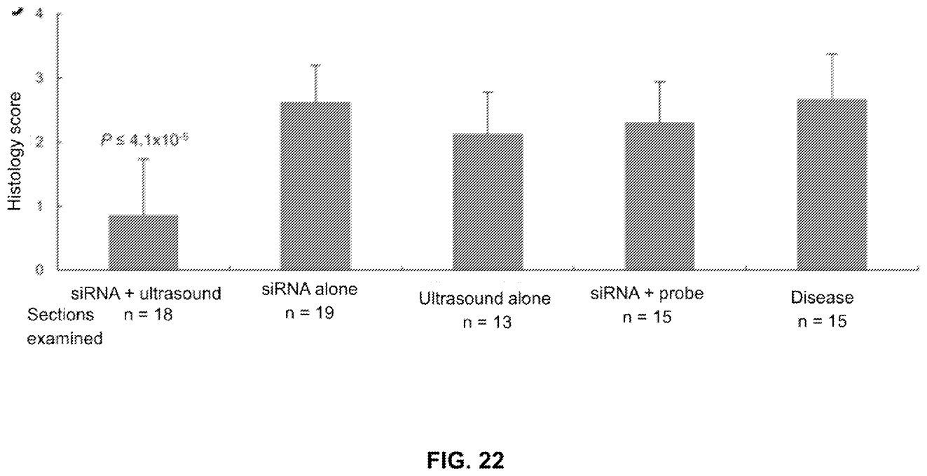

FIG. 22 is graph showing histology scores of colonic tissue sections on Day 8. P-values were determined by one-way ANOVA with multiple comparisons. Data represent averages+one standard deviation.

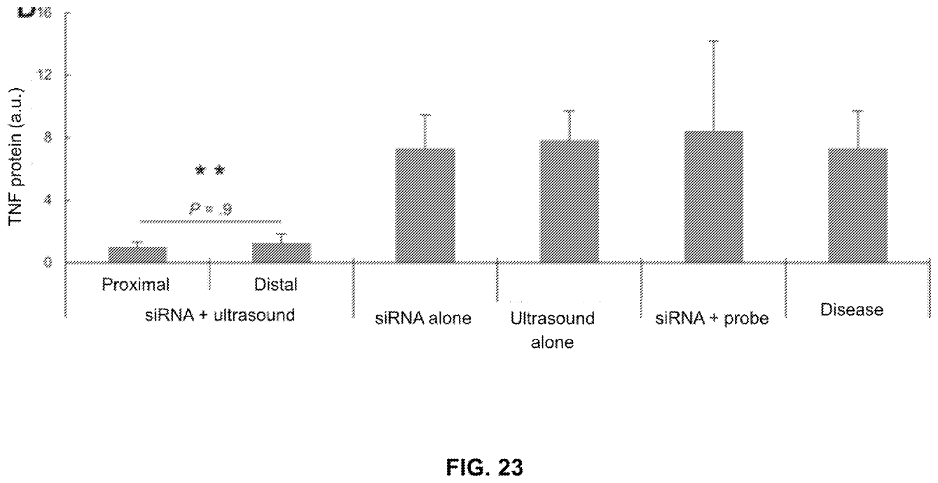

FIG. 23 is a graph showing TNF protein levels from colonic tissue biopsies normalized by total protein content on Day 8. ** represents P<0.014 for both siRNA+ultrasound groups compared to any other group (determined by one-way ANOVA with multiple comparisons). P-values were determined by one-way ANOVA with multiple comparisons. Data represent averages+one standard deviation.

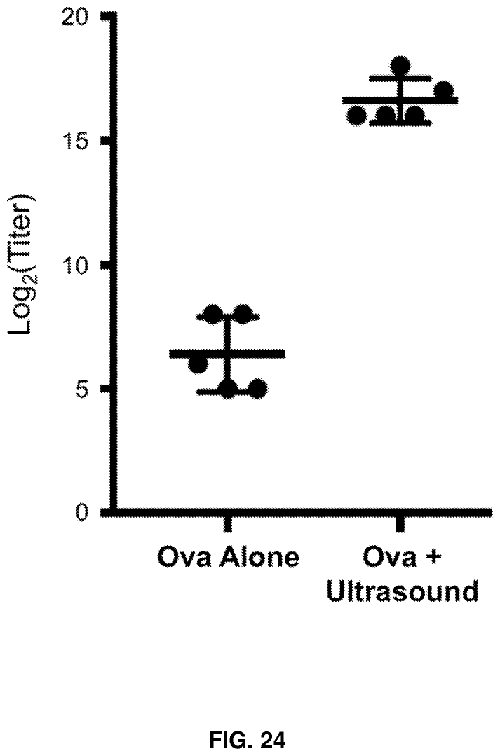

FIG. 24 is a graph showing ova IgG titer levels 7-weeks after administration of ova alone or in combination with ultrasound. Titer level is reported on a log-2 basis. P<0.05.

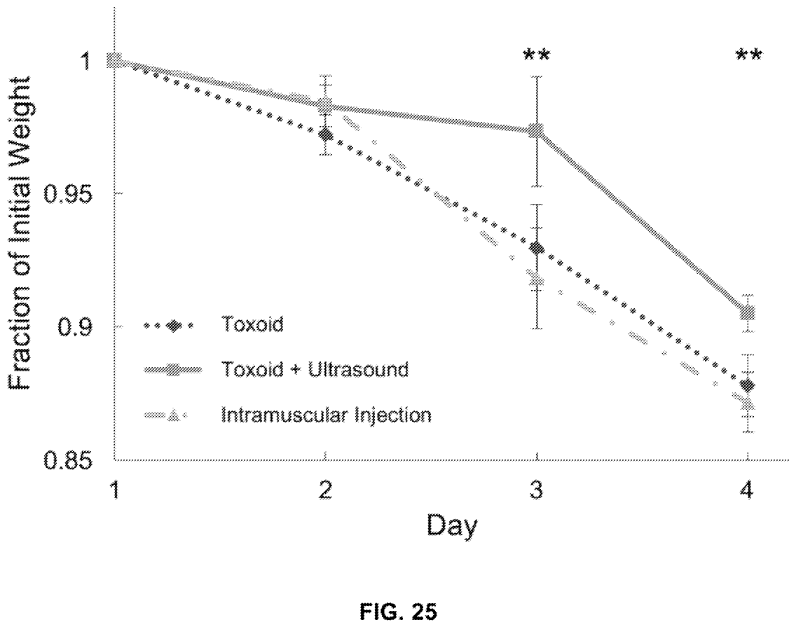

FIG. 25 is a graph showing animal body weight after challenge with a lethal dose of C. diff spores on Day 1 normalized to the animals' weight pre-challenge. Error bars represent averages +/−1 SD. ** represents P<0.05 between the Toxoid+Ultrasound group and all other groups determined by a one-way ANOVA with multiple comparisons.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A device for delivery of an agent to an internal tissue of a subject, the device comprising:
an ultrasound transducer configured to fit within a lumen of an endoscope; and
an electrical conductor that operably couples the ultrasound transducer to a power source, the electrical conductor configured to fit at least partly within the lumen of the endoscope.

2. The device of claim 1, wherein the ultrasound transducer has a maximum diameter of not more than 20 mm.

3. The device of claim 1, further comprising a cover enclosing a portion of the ultrasound transducer.

4. The device of claim 3, wherein the cover comprises an acoustically transparent material.

5. The device of claim 3, wherein the cover comprises a material that is resistant to degradation by one or more of acid and gastric enzymes.

6. The device of claim 1, wherein the electrical conductor comprises a length of at least 400 mm.

7. The device of claim 6, wherein a distal end of the electrical conductor contacts the transducer and a proximal end of the electrical conductor is configured to contact the power source.

8. The device of claim 1, further comprising a tube comprising a distal end proximate the ultrasound transducer and a proximal end configured to contact a fluid source containing the fluid.

9. The device of claim 8, wherein the tube is configured to fit at least partly within the lumen of the endoscope.

10. The device of claim 9, wherein the distal end of the tube contacts the transducer.

11. A device for delivery of an agent to an internal tissue of a subject, the device comprising:
an ultrasound transducer configured to fit into an esophagus of a human; and
an electrical conductor that operably couples the transducer to a power source.

12. The device of claim 11, wherein the electrical conductor is configured to fit at least partly within a lumen of an endoscope.

13. The device of claim 11, further comprising a cover enclosing a portion of the ultrasound transducer.

14. The device of claim 13, wherein the cover comprises an acoustically transparent material.

15. The device of claim 13, wherein the cover comprises a material that is resistant to degradation by one or more of acid and gastric enzymes.

16. The device of claim 11, wherein the electrical conductor comprises a length of at least 400 mm.

17. The device of claim 16, wherein a distal end of the electrical conductor contacts the transducer and a proximal end of the electrical conductor is configured to contact the power source.

18. The device of claim 11, further comprising a tube comprising a distal end proximate the ultrasound transducer and a proximal end configured to contact a fluid source containing the fluid.

19. The device of claim 18, wherein the tube is configured to fit at least partly within the lumen of the endoscope.

20. The device of claim 18, wherein the distal end of the tube contacts the transducer.

21-30. (canceled)
